SEQUENCE LISTING (121 sequences)

SEQ ID NO: 1 - DNA, 1000 bp, synthetic construct
  misc_feature 1..1000: note = Segment A
  source 1..1000: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccattctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcg

SEQ ID NO: 2 - DNA, 1000 bp, synthetic construct
  misc_feature 1..1000: note = Segment B
  source 1..1000: mol_type = other DNA
acttcaaggaggacggcaacattctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctcaaactcaagcttcgaattctgcagtcgacggtaccgcgggcccgggacccaccggatcaagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaaggga

SEQ ID NO: 3 - DNA, 1000 bp, synthetic construct
  misc_feature 1..1000: note = Segment C
  source 1..1000: mol_type = other DNA
agaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccaacccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacagtgaggatcgtttcgcgccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgc

SEQ ID NO: 4 - DNA, 1000 bp, synthetic construct
  misc_feature 1..1000: note = Segment D
  source 1..1000: mol_type = other DNA
atacgcttgatccggctacctgcccattcgaccaccaagcgaaacatcgcatcgagcgagcacgtactcgcatggaagccggtcttgtcgatcaggacgatctggacgaggagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaggagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggtgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgt

SEQ ID NO: 5 - DNA, 500 bp, synthetic construct
  misc_feature 1..500: note = Segment E
  source 1..500: mol_type = other DNA
tacgacattttggaaagtcccgttgattttggtgccaaaacaaactcccattgacgtcaatggggtggagacttggaaatccccgtgagtcaaaccgctatccacgcccattgatgtactgccaaaaccgcatcaccatggtaatagcgatgactaatacgtagatgtactgccaagtaggaaagtcccataaggtcatgtactgggcataatgccaggcgggccatttaccgtcattgacgtcaatagggggcgtacttggcatatgatacacttgatgtactgccaagtgggcagtttaccgtaaatactccacccattgacgtcaatggaaagtccctattggcgttactatgggaacatacgtcattattgacgtcaatgggcgggggtcgttgggcggtcagccaggcgggccatttaccgtaagttatgtaacgcggaactccatatatgggctatgaactaatgaccccgtaattgattactattaataacta

SEQ ID NO: 6 - DNA, 1000 bp, synthetic construct
  misc_feature 1..1000: note = Segment F
  source 1..1000: mol_type = other DNA
cattttatgtttcaggttcagggggaggtgtgggaggttttttaaagcaagtaaaacctctacaaatgtggtatggctgattatgatcagttatcttgatccggtgggtcccgggcccgcggtaccgtcgactgcagaattcgaagcttgagctcaaatctgagtccggacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccagaatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaacttcagggtcagcttgccgtaggtggcatcgccctcgccctcgccggacacgctgaacttgtggccgtttacgtcgccgtccagctcgaccagaatgggcaccaccccggtgaacagctcctcgcccttgctcaccatggtggcgaccggtagcgctagcggatctgacggttcactaaaccagctctgcttatatagacctcccaccgtacacgcctaccgcccatttgcgtcaatggggcggagttgtt

SEQ ID NO: 7 - DNA, 1000 bp, synthetic construct
  misc_feature 1..1000: note = Segment G
  source 1..1000: mol_type = other DNA
aaattagtcagccatggggcggagaatgggcggaactgggcggagttaggggcgggttgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctggttctttccgcctcaggactcttcctttttcaatattattgaagcatttatcagggttattgtctcatgagcggatacatatttgaatgtatttagaaaaataaacaaataggggttccgcgcacatttccccgaaaagtgccacctgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgcttacaatttacgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcatt

SEQ ID NO: 8 - DNA, 1000 bp, synthetic construct
  misc_feature 1..1000: note = Segment H
  source 1..1000: mol_type = other DNA
cggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcctcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcctcgtccagatcgtcctgatcgacaagaccggcttccatgcgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatggtggcgcgaaacgatcctcactgtctcttgatcgatctttgcaaaagcctaggcctccaaaaaagcctcctcactacttctggaatagctcagaggccgaggcggcctcggcctctgcataaataaaaa

SEQ ID NO: 9 - DNA, 500 bp, synthetic construct
  misc_feature 1..500: note = Segment I
  source 1..500: mol_type = other DNA
acgctcagtggaacgaaaactcacgttaagggattttggtcatgagattatcaaaaaggatcttcacctagatccttttaaattaaaaatgaagttttaaatcaatctaaagtatatatgagtaacctgaggctatggcagggcctgccgccccgacgttggctgcgagccctgggccttcacccgaacttggggggtggggtggggaaaaggaagaaacgcgggcgtattggccccaatggggtctcggtggggtatcgacagagtgccagccctgggaccgaaccccgcgtttatgaacaaacgacccaacaccgtgcgttttattctgtctttttattgccgtcatagcgcgggttccttccggtattgtctccttccgtgtttcagttagcctccccctagggtgggcgaagaactccagcatgagatccccgcgctggaggatcaccagccggcgtcccggaaaacgattccgaagcccaacctttcatagaagg

SEQ ID NO: 10 - DNA, 1001 bp, synthetic construct
  misc_feature 1..1001: note = Segment J
  source 1..1001: mol_type = other DNA
gaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccaacccgcccctaactccgcccagttccgcccattctccgccccatggctgactaattt

SEQ ID NO: 11 - DNA, 1001 bp, synthetic construct
  misc_feature 1..1001: note = Segment K
  source 1..1001: mol_type = other DNA
tgatttataagggattttgccgatttcggcctattggttaaaaaatgagctgatttaacaaaaatttaacgcgaattttaacaaaatattaacgcttacaatttacgcgttaagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtatggctgattatgatcagttatcttgatccggtgggtcccgggcccgcggtaccgtcgactgcagaattcgaagcttgagctttagatctgagtccggacttgtacagctcgtccatgccgagagtgatcccggcggcggtcacgaactccagcaggaccatgtgatcgcgcttctcgttggggtctttgctcagggcggactgggtgctcaggtagtggttgtcgggcagcagcacggggccgtcgccgatgggggtgttctgctggtagtggtcggcgagctgcacgctgccgtcctcgatgttgtggcggatcttgaagttcaccttgatgccgttcttctgcttgtcggccatgatatagacgttgtggctgttgtagttgtactccagcttgtgccccagaatgttgccgtcctccttgaagtcgatgcccttcagctcgatgcggttcaccagggtgtcgccctcgaacttcacctcggcgcgggtcttgtagttgccgtcgtccttgaagaagatggtgcgctcctggacgtagccttcgggcatggcggacttgaagaagtcgtgctgcttcatgtggtcggggtagcggctgaagcactgcacgccgtaggtcagggtggtcacgagggtgggccagggcacgggcagcttgccggtggtgcagatgaac

SEQ ID NO: 12 - DNA, 1001 bp, synthetic construct
  misc_feature 1..1001: note = Segment L
  source 1..1001: mol_type = other DNA
atgaatccagaaaagcggccattttccaccatgatattcggcaagcaggcatcgccatgggtcacgacgagatcctcgccgtcgggcatgctcgccttgagcctggcgaacagttcggctggcgcgagcccctgatgctcctcgtccagatcgtcctgatcgacaagaccggcttccatgcgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcttgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatggtggcgcgaaacgatcctcactgtctcttgatcgatctttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggttgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgg

SEQ ID NO: 13 - DNA, 200 bp, synthetic construct
  misc_feature 1..200: note = Segment M
  source 1..200: mol_type = other DNA
ttgatgccgttcttctgcttgtcggccatgatatagacgttgtggctgttgtagttgtactccagcttgtgccccagaatgttgccgtcctccttgaagtcgatgcccttcagctcgatgcggttcaccagggtgtcgccctcgaacttcacctcggcgcgggtcttgtagttgccgtcgtccttgaagaagatggtgcg

SEQ ID NO: 14 - DNA, 200 bp, synthetic construct
  misc_feature 1..200: note = Segment N
  source 1..200: mol_type = other DNA
gcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccc

SEQ ID NO: 15 - DNA, 200 bp, synthetic construct
  misc_feature 1..200: note = Segment O
  source 1..200: mol_type = other DNA
tcgtcctgatcgacaagaccggcttccatgcgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagcca

SEQ ID NO: 16 - DNA, 200 bp, synthetic construct
  misc_feature 1..200: note = Segment P
  source 1..200: mol_type = other DNA
acgctcagtggaacgaaaactcacgttaagggattttggtcatgagattatcaaaaaggatcttcacctagatccttttaaattaaaaatgaagttttaaatcaatctaaagtatatatgagtaacctgaggctatggcagggcctgccgccccgacgttggctgcgagccctgggccttcacccgaacttggggggtgg

SEQ ID NO: 17 - DNA, 4000 bp, synthetic construct
  misc_feature 1..4000: note = Synthetic gene
  source 1..4000: mol_type = other DNA
tagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatccgctagcgctaccggtcgccaccatggtgagcaagggcgaggagctgttcaccggggtggtgcccattctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacattctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtccggactcagatctaaagctcaagcttcgaattctgcagtcgacggtaccgcgggcccgggacccaccggatcaagataactgatcataatcagccataccacatttgtagaggttttacttgctttaaaaaacctcccacacctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttaacgcgtaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtcctgaggcggaaagaaccagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccaacccgcccctaactccgcccagttccgcccattctccgccccatggctgactaattttttttatttatgcagaggccgaggccgcctcggcctctgagctattccagaagtagtgaggaggcttttttggaggcctaggcttttgcaaagatcgatcaagagacagtgaggatcgtttcgcgccaccatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcgcatggaagccggtcttgtcgatcaggacgatctggacgaggagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaggagcttggcggcgaatgggctgaccgcttcctcgtgctttacggtatcgccgctcccgattcgcagcgcatcgccttctatcgccttcttgacgagttcttctgagcgggactctggggttcgaaatgaccgaccaagcgacgcccaacctgccatcacgagatttcgattccaccgccgccttctatgaaaggttgggcttcggaatcgttttccgggacgccggctggtgatcctccagcgcggggatctcatgctggagttcttcgcccaccctagggggaggctaactgaaacacggaaggagacaataccggaaggaacccgcgctatgacggcaataaaaagacagaataaaacgcacggtgttgggtcgtttgttcataaacgcggggttcggtcccagggctggcactctgtcgataccccaccgagaccccattggggccaatacgcccgcgtttcttccttttccccaccccaccccccaagttcgggtgaaggcccagggctcgcagccaacgtcggggcggcaggccctgccatagcctcaggttactcatatatactttagattgatttaaaacttcatttttaatttaaaaggatctaggtgaagatcctttttgataatctcatgaccaaaatcccttaacgtgagttttcgttccactgagcgt

SEQ ID NO: 18 - DNA, 60 bp, synthetic construct
  source 1..60: mol_type = other DNA; note = FWD1
tagttattaatagtaatcaattacggggtcattagttcatagcccatatatggagttccg

SEQ ID NO: 19 - DNA, 60 bp, synthetic construct
  source 1..60: mol_type = other DNA; note = FWD2
atggcccgcctggctgaccgcccaacgacccccgcccattgacgtcaataatgacgtatg

SEQ ID NO: 20 - DNA, 60 bp, synthetic construct
  source 1..60: mol_type = other DNA; note = FWD3
gggactttccattgacgtcaatgggtggagtatttacggtaaactgcccacttggcagta

SEQ ID NO: 21 - DNA, 60 bp, synthetic construct
  source 1..60: mol_type = other DNA; note = FWD4
aagtacgccccctattgacgtcaatgacggtaaatggcccgcctggcattatgcccagta

SEQ ID NO: 22 - DNA, 60 bp, synthetic construct
  source 1..60: mol_type = other DNA; note = FWD5
ctacttggcagtacatctacgtattagtcatcgctattaccatggtgatgcggttttggc

SEQ ID NO: 23 - DNA, 60 bp, synthetic construct
  source 1..60: mol_type = other DNA; note = FWD6
tagcggtttgactcacggggatttccaagtctccaccccattgacgtcaatgggagtttg

SEQ ID NO: 24 - DNA, 60 bp, synthetic construct
  source 1..60: mol_type = other DNA; note = FWD7
ggactttccaaaatgtcgtaacaactccgccccattgacgcaaatgggcggtaggcgtgt

SEQ ID NO: 25 - DNA, 60 bp, synthetic construct
  source 1..60: mol_type = other DNA; note = FWD8
gcagagctggtttagtgaaccgtcagatccgctagcgctaccggtcgccaccatggtgag

SEQ ID NO: 26 - DNA, 60 bp, synthetic construct
  source 1..60: mol_type = other DNA; note = FWD9
ccggggtggtgcccattctggtcgagctggacggcgacgtaaacggccacaagttcagcg

SEQ ID NO: 27 - DNA, 60 bp, synthetic construct
  source 1..60: mol_type = other DNA; note = FWD10
gatgccacctacggcaagctgaccctgaagttcatctgcaccaccggcaagctgcccgtg

SEQ ID NO: 28 - DNA, 60 bp, synthetic construct
  source 1..60: mol_type = other DNA; note = FWD11
caccctgacctacggcgtgcagtgcttcagccgctaccccgaccacatgaagcagcacga

SEQ ID NO: 29 - DNA, 60 bp, synthetic construct
  source 1..60: mol_type = other DNA; note = FWD12
ccgaaggctacgtccaggagcgcaccatcttcttcaaggacgacggcaactacaagaccc

SEQ ID NO: 30 - DNA, 60 bp, synthetic construct
  source 1..60: mol_type = other DNA; note = FWD13
ggcgacaccctggtgaaccgcatcgagctgaagggcatcgacttcaaggaggacggcaac

SEQ ID NO: 31 - DNA, 60 bp, synthetic construct
  source 1..60: mol_type = other DNA; note = FWD14
gtacaactacaacagccacaacgtctatatcatggccgacaagcagaagaacggcatcaa

SEQ ID NO: 32 - DNA, 60 bp, synthetic construct
  source 1..60: mol_type = other DNA; note = FWD15
acaacatcgaggacggcagcgtgcagctcgccgaccactaccagcagaacacccccatcg

SEQ ID NO: 33 - DNA, 60 bp, synthetic construct
  source 1..60: mol_type = other DNA; note = FWD16
cccgacaaccactacctgagcacccagtccgccctgagcaaagaccccaacgagaagcgc

SEQ ID NO: 34 - DNA, 60 bp, synthetic construct
  source 1..60: mol_type = other DNA; note = FWD17
gttcgtgaccgccgccgggatcactctcggcatggacgagctgtacaagtccggactcag

SEQ ID NO: 35 - DNA, 60 bp, synthetic construct
  source 1..60: mol_type = other DNA; note = FWD18
attctgcagtcgacggtaccgcgggcccgggatccaccggatctagataactgatcataa

SEQ ID NO: 36 - DNA, 60 bp, synthetic construct
  source 1..60: mol_type = other DNA; note = FWD19
gaggttttacttgctttaaaaaacctcccacacctccccctgaacctgaaacataaaatg

SEQ ID NO: 37 - DNA, 60 bp, synthetic construct
  source 1..60: mol_type = other DNA; note = FWD20
cttgtttattgcagcttataatggttacaaataaagcaatagcatcacaaatttcacaaa

SEQ ID NO: 38 - DNA, 60 bp, synthetic construct
  source 1..60: mol_type = other DNA; note = FWD21
attctagttgtggtttgtccaaactcatcaatgtatcttaacgcgtaaattgtaagcgtt

SEQ ID NO: 39 - DNA, 60 bp, synthetic construct
  source 1..60: mol_type = other DNA; note = FWD22
gttaaatttttgttaaatcagctcattttttaaccaataggccgaaatcggcaaaatccc

SEQ ID NO: 40 - DNA, 60 bp, synthetic construct
  source 1..60: mol_type = other DNA; note = FWD23
ccgagatagggttgagtgttgttccagtttggaacaagagtccactattaaagaacgtgg

SEQ ID NO: 41 - DNA, 60 bp, synthetic construct
  source 1..60: mol_type = other DNA; note = FWD24
aaaaccgtctatcagggcgatggcccactacgtgaaccatcaccctaatcaagttttttg

SEQ ID NO: 42 - DNA, 60 bp, synthetic construct
  source 1..60: mol_type = other DNA; note = FWD25
actaaatcggaaccctaaagggagcccccgatttagagcttgacggggaaagccggcgaa

SEQ ID NO: 43 - DNA, 60 bp, synthetic construct
  source 1..60: mol_type = other DNA; note = FWD26
agaaagcgaaaggagcgggcgctagggcgctggcaagtgtagcggtcacgctgcgcgtaa

SEQ ID NO: 44 - DNA, 60 bp, synthetic construct
  source 1..60: mol_type = other DNA; note = FWD27
aatgcgccgctacagggcgcgtcaggtggcacttttcggggaaatgtgcgcggaacccct

SEQ ID NO: 45 - DNA, 60 bp, synthetic construct
  source 1..60: mol_type = other DNA; note = FWD28
acattcaaatatgtatccgctcatgagacaataaccctgataaatgcttcaataatattg

SEQ ID NO: 46 - DNA, 60 bp, synthetic construct
  source 1..60: mol_type = other DNA; note = FWD29
cggaaagaaccagctgtggaatgtgtgtcagttagggtgtggaaagtccccaggctcccc

SEQ ID NO: 47 - DNA, 60 bp, synthetic construct
  source 1..60: mol_type = other DNA; note = FWD30
gcatgcatctcaattagtcagcaaccaggtgtggaaagtccccaggctccccagcaggca

SEQ ID NO: 48 - DNA, 60 bp, synthetic construct
  source 1..60: mol_type = other DNA; note = FWD31
ctcaattagtcagcaaccatagtcccgcccctaactccgcccaacccgcccctaactccg

SEQ ID NO: 49 - DNA, 60 bp, synthetic construct
  source 1..60: mol_type = other DNA; note = FWD32
gccccatggctgactaattttttttatttatgcagaggccgaggccgcctcggcctctga

SEQ ID NO: 50 - DNA, 60 bp, synthetic construct
  source 1..60: mol_type = other DNA; note = FWD33
gaggcttttttggaggcctaggcttttgcaaagatcgatcaagagacagtgaggatcgtt

SEQ ID NO: 51 - DNA, 60 bp, synthetic construct
  source 1..60: mol_type = other DNA; note = FWD34
aagatggattgcacgcaggttctccggccgcttgggtggagaggctattcggctatgact

SEQ ID NO: 52 - DNA, 60 bp, synthetic construct
  source 1..60: mol_type = other DNA; note = FWD35
tgctctgatgccgccgtgttccggctgtcagcgcaggggcgcccggttctttttgtcaag

SEQ ID NO: 53 - DNA, 60 bp, synthetic construct
  source 1..60: mol_type = other DNA; note = FWD36
gaatgaactgcaagacgaggcagcgcggctatcgtggctggccacgacgggcgttccttg

SEQ ID NO: 54 - DNA, 60 bp, synthetic construct
  source 1..60: mol_type = other DNA; note = FWD37
tcactgaagcgggaagggactggctgctattgggcgaagtgccggggcaggatctcctgt

SEQ ID NO: 55 - DNA, 60 bp, synthetic construct
  source 1..60: mol_type = other DNA; note = FWD38
gagaaagtatccatcatggctgatgcaatgcggcggctgcatacgcttgatccggctacc

SEQ ID NO: 56 - DNA, 60 bp, synthetic construct
  source 1..60: mol_type = other DNA; note = FWD39
gaaacatcgcatcgagcgagcacgtactcgcatggaagccggtcttgtcgatcaggacga

SEQ ID NO: 57 - DNA, 60 bp, synthetic construct
  source 1..60: mol_type = other DNA; note = FWD40
ggctcgcgccagccgaactgttcgccaggctcaaggcgagcatgcccgacggcgaggatc

SEQ ID NO: 58 - DNA, 60 bp, synthetic construct
  source 1..60: mol_type = other DNA; note = FWD41
gcctgcttgccgaatatcatggtggaaaatggccgcttttctggattcatcgactgtggc

SEQ ID NO: 59 - DNA, 60 bp, synthetic construct
  source 1..60: mol_type = other DNA; note = FWD42
ctatcaggacatagcgttggctacccgtgatattgctgaggagcttggcggcgaatgggc

SEQ ID NO: 60 - DNA, 60 bp, synthetic construct
  source 1..60: mol_type = other DNA; note = FWD43
acggtatcgccgctcccgattcgcagcgcatcgccttctatcgccttcttgacgagttct

SEQ ID NO: 61 - DNA, 60 bp, synthetic construct
  source 1..60: mol_type = other DNA; note = FWD44
tcgaaatgaccgaccaagcgacgcccaacctgccatcacgagatttcgattccaccgccg

SEQ ID NO: 62 - DNA, 60 bp, synthetic construct
  source 1..60: mol_type = other DNA; note = FWD45
ttcggaatcgttttccgggacgccggctggtgatcctccagcgcggggatctcatgctgg

SEQ ID NO: 63 - DNA, 60 bp, synthetic construct
  source 1..60: mol_type = other DNA; note = FWD46
gggaggctaactgaaacacggaaggagacaataccggaaggaacccgcgctatgacggca

SEQ ID NO: 64 - DNA, 60 bp, synthetic construct
  source 1..60: mol_type = other DNA; note = FWD47
gcacggtgttgggtcgtttgttcataaacgcggggttcggtcccagggctggcactctgt

SEQ ID NO: 65 - DNA, 60 bp, synthetic construct
  source 1..60: mol_type = other DNA; note = FWD48
attggggccaatacgcccgcgtttcttccttttccccaccccaccccccaagttcgggtg

SEQ ID NO: 66 - DNA, 60 bp, synthetic construct
  source 1..60: mol_type = other DNA; note = FWD49
aacgtcggggcggcaggccctgccatagcctcaggttactcatatatactttagattgat

SEQ ID NO: 67 - DNA, 60 bp, synthetic construct
  source 1..60: mol_type = other DNA; note = FWD50
taaaaggatctaggtgaagatcctttttgataatctcatgaccaaaatcccttaacgtga

SEQ ID NO: 68 - DNA, 40 bp, synthetic construct
  source 1..40: mol_type = other DNA; note = RVS1
acgctcagtggaacgaaaactcacgttaagggattttggt

SEQ ID NO: 69 - DNA, 60 bp, synthetic construct
  source 1..60: mol_type = other DNA; note = RVS2
tcttcacctagatccttttaaattaaaaatgaagttttaaatcaatctaaagtatatatg

SEQ ID NO: 70 - DNA, 60 bp, synthetic construct
  source 1..60: mol_type = other DNA; note = RVS3
gggcctgccgccccgacgttggctgcgagccctgggccttcacccgaacttggggggtgg

SEQ ID NO: 71 - DNA, 60 bp, synthetic construct
  source 1..60: mol_type = other DNA; note = RVS4
gcgggcgtattggccccaatggggtctcggtggggtatcgacagagtgccagccctggga

SEQ ID NO: 72 - DNA, 60 bp, synthetic construct
  source 1..60: mol_type = other DNA; note = RVS5
caaacgacccaacaccgtgcgttttattctgtctttttattgccgtcatagcgcgggttc

SEQ ID NO: 73 - DNA, 60 bp, synthetic construct
  source 1..60: mol_type = other DNA; note = RVS6
cgtgtttcagttagcctccccctagggtgggcgaagaactccagcatgagatccccgcgc

SEQ ID NO: 74 - DNA, 60 bp, synthetic construct
  source 1..60: mol_type = other DNA; note = RVS7
tcccggaaaacgattccgaagcccaacctttcatagaaggcggcggtggaatcgaaatct

SEQ ID NO: 75 - DNA, 60 bp, synthetic construct
  source 1..60: mol_type = other DNA; note = RVS8
cgcttggtcggtcatttcgaaccccagagtcccgctcagaagaactcgtcaagaaggcga

SEQ ID NO: 76 - DNA, 60 bp, synthetic construct
  source 1..60: mol_type = other DNA; note = RVS9
atcgggagcggcgataccgtaaagcacgaggaagcggtcagcccattcgccgccaagctc

SEQ ID NO: 77 - DNA, 60 bp, synthetic construct
  source 1..60: mol_type = other DNA; note = RVS10
ccaacgctatgtcctgatagcggtccgccacacccagccggccacagtcgatgaatccag

SEQ ID NO: 78 - DNA, 60 bp, synthetic construct
  source 1..60: mol_type = other DNA; note = RVS11
atgatattcggcaagcaggcatcgccatgggtcacgacgagatcctcgccgtcgggcatg

SEQ ID NO: 79 - DNA, 60 bp, synthetic construct
  source 1..60: mol_type = other DNA; note = RVS12
cagttcggctggcgcgagcccctgatgctcctcgtccagatcgtcctgatcgacaagacc

SEQ ID NO: 80 - DNA, 60 bp, synthetic construct
  source 1..60: mol_type = other DNA; note = RVS13
ctcgctcgatgcgatgtttcgcttggtggtcgaatgggcaggtagccggatcaagcgtat

SEQ ID NO: 81 - DNA, 60 bp, synthetic construct
  source 1..60: mol_type = other DNA; note = RVS14
gccatgatggatactttctcggcaggagcaaggtgagatgacaggagatcctgccccggc

SEQ ID NO: 82 - DNA, 60 bp, synthetic construct
  source 1..60: mol_type = other DNA; note = RVS15
gtcccttcccgcttcagtgacaacgtcgagcacagctgcgcaaggaacgcccgtcgtggc

SEQ ID NO: 83 - DNA, 60 bp, synthetic construct
  source 1..60: mol_type = other DNA; note = RVS16
cctcgtcttgcagttcattcagggcaccggacaggtcggtcttgacaaaaagaaccgggc

SEQ ID NO: 84 - DNA, 60 bp, synthetic construct
  source 1..60: mol_type = other DNA; note = RVS17
aacacggcggcatcagagcagccgattgtctgttgtgcccagtcatagccgaatagcctc

SEQ ID NO: 85 - DNA, 60 bp, synthetic construct
  source 1..60: mol_type = other DNA; note = RVS18
acctgcgtgcaatccatcttgttcaatcatggtggcgcgaaacgatcctcactgtctctt

SEQ ID NO: 86 - DNA, 60 bp, synthetic construct
  source 1..60: mol_type = other DNA; note = RVS19
taggcctccaaaaaagcctcctcactacttctggaatagctcagaggccgaggcggcctc

SEQ ID NO: 87 - DNA, 60 bp, synthetic construct
  source 1..60: mol_type = other DNA; note = RVS20
aaattagtcagccatggggcggagaatgggcggaactgggcggagttaggggcgggttgg

SEQ ID NO: 88 - DNA, 60 bp, synthetic construct
  source 1..60: mol_type = other DNA; note = RVS21
atggttgctgactaattgagatgcatgctttgcatacttctgcctgctggggagcctggg

SEQ ID NO: 89 - DNA, 60 bp, synthetic construct
  source 1..60: mol_type = other DNA; note = RVS22
tgactaattgagatgcatgctttgcatacttctgcctgctggggagcctggggactttcc

SEQ ID NO: 90 - DNA, 60 bp, synthetic construct
  source 1..60: mol_type = other DNA; note = RVS23
tccacagctggttctttccgcctcaggactcttcctttttcaatattattgaagcattta

SEQ ID NO: 91 - DNA, 60 bp, synthetic construct
  source 1..60: mol_type = other DNA; note = RVS24
gcggatacatatttgaatgtatttagaaaaataaacaaataggggttccgcgcacatttc

SEQ ID NO: 92 - DNA, 60 bp, synthetic construct
  source 1..60: mol_type = other DNA; note = RVS25
gcgccctgtagcggcgcattaagcgcggcgggtgtggtggttacgcgcagcgtgaccgct

SEQ ID NO: 93 - DNA, 60 bp, synthetic construct
  source 1..60: mol_type = other DNA; note = RVS26
gcccgctcctttcgctttcttcccttcctttctcgccacgttcgccggctttccccgtca

SEQ ID NO: 94 - DNA, 60 bp, synthetic construct
  source 1..60: mol_type = other DNA; note = RVS27
ctttagggttccgatttagtgctttacggcacctcgaccccaaaaaacttgattagggtg

SEQ ID NO: 95 - DNA, 60 bp, synthetic construct
  source 1..60: mol_type = other DNA; note = RVS28
tcgccctgatagacggtttttcgccctttgacgttggagtccacgttctttaatagtgga

SEQ ID NO: 96 - DNA, 60 bp, synthetic construct
  source 1..60: mol_type = other DNA; note = RVS29
aacactcaaccctatctcggtctattcttttgatttataagggattttgccgatttcggc

SEQ ID NO: 97 - DNA, 60 bp, synthetic construct
  source 1..60: mol_type = other DNA; note = RVS30
tgatttaacaaaaatttaacgcgaattttaacaaaatattaacgcttacaatttacgcgt

SEQ ID NO: 98 - DNA, 60 bp, synthetic construct
  source 1..60: mol_type = other DNA; note = RVS31
ggacaaaccacaactagaatgcagtgaaaaaaatgctttatttgtgaaatttgtgatgct

SEQ ID NO: 99 - DNA, 60 bp, synthetic construct
  source 1..60: mol_type = other DNA; note = RVS32
tataagctgcaataaacaagttaacaacaacaattgcattcattttatgtttcaggttca

SEQ ID NO: 100 - DNA, 60 bp, synthetic construct
  source 1..60: mol_type = other DNA; note = RVS33
tttaaagcaagtaaaacctctacaaatgtggtatggctgattatgatcagttatctagat

SEQ ID NO: 101 - DNA, 60 bp, synthetic construct
  source 1..60: mol_type = other DNA; note = RVS34
ggtaccgtcgactgcagaattcgaagcttgagctttagatctgagtccggacttgtacag

SEQ ID NO: 102 - DNA, 60 bp, synthetic construct
  source 1..60: mol_type = other DNA; note = RVS35
tcccggcggcggtcacgaactccagcaggaccatgtgatcgcgcttctcgttggggtctt

SEQ ID NO: 103 - DNA, 60 bp, synthetic construct
  source 1..60: mol_type = other DNA; note = RVS36
ctcaggtagtggttgtcgggcagcagcacggggccgtcgccgatgggggtgttctgctgg

SEQ ID NO: 104 - DNA, 60 bp, synthetic construct
  source 1..60: mol_type = other DNA; note = RVS37
gctgccgtcctcgatgttgtggcggatcttgaagttcaccttgatgccgttcttctgctt

SEQ ID NO: 105 - DNA, 60 bp, synthetic construct
  source 1..60: mol_type = other DNA; note = RVS38
tgtggctgttgtagttgtactccagcttgtgccccagaatgttgccgtcctccttgaagt

SEQ ID NO: 106 - DNA, 60 bp, synthetic construct
  source 1..60: mol_type = other DNA; note = RVS39
cggttcaccagggtgtcgccctcgaacttcacctcggcgcgggtcttgtagttgccgtcg

SEQ ID NO: 107 - DNA, 60 bp, synthetic construct
  source 1..60: mol_type = other DNA; note = RVS40
ctcctggacgtagccttcgggcatggcggacttgaagaagtcgtgctgcttcatgtggtc

SEQ ID NO: 108 - DNA, 60 bp, synthetic construct
  source 1..60: mol_type = other DNA; note = RVS41
gcacgccgtaggtcagggtggtcacgagggtgggccagggcacgggcagcttgccggtgg

SEQ ID NO: 109 - DNA, 60 bp, synthetic construct
  source 1..60: mol_type = other DNA; note = RVS42
agcttgccgtaggtggcatcgccctcgccctcgccggacacgctgaacttgtggccgttt

SEQ ID NO: 110 - DNA, 60 bp, synthetic construct
  source 1..60: mol_type = other DNA; note = RVS43
cagaatgggcaccaccccggtgaacagctcctcgcccttgctcaccatggtggcgaccgg

SEQ ID NO: 111 - DNA, 60 bp, synthetic construct
  source 1..60: mol_type = other DNA; note = RVS44
gttcactaaaccagctctgcttatatagacctcccaccgtacacgcctaccgcccatttg

SEQ ID NO: 112 - DNA, 60 bp, synthetic construct
  source 1..60: mol_type = other DNA; note = RVS45
tacgacattttggaaagtcccgttgattttggtgccaaaacaaactcccattgacgtcaa

SEQ ID NO: 113 - DNA, 60 bp, synthetic construct
  source 1..60: mol_type = other DNA; note = RVS46
ccccgtgagtcaaaccgctatccacgcccattgatgtactgccaaaaccgcatcaccatg

SEQ ID NO: 114 - DNA, 60 bp, synthetic construct
  source 1..60: mol_type = other DNA; note = RVS47
gtagatgtactgccaagtaggaaagtcccataaggtcatgtactgggcataatgccaggc

SEQ ID NO: 115 - DNA, 60 bp, synthetic construct
  source 1..60: mol_type = other DNA; note = RVS48
cgtcaatagggggcgtacttggcatatgatacacttgatgtactgccaagtgggcagttt

SEQ ID NO: 116 - DNA, 60 bp, synthetic construct
  source 1..60: mol_type = other DNA; note = RVS49
tgacgtcaatggaaagtccctattggcgttactatgggaacatacgtcattattgacgtc

SEQ ID NO: 117 - DNA, 60 bp, synthetic construct
  source 1..60: mol_type = other DNA; note = RVS50
cggtcagccaggcgggccatttaccgtaagttatgtaacgcggaactccatatatgggct

SEQ ID NO: 118 - DNA, 50 bp, synthetic construct
  source 1..50: mol_type = other DNA; note = Short ssDNA molecule
agaatgttgccgtcctccttgaagtcgatgcccttcagctcgatgcggtt

SEQ ID NO: 119 - DNA, 50 bp, synthetic construct
  source 1..50: mol_type = other DNA; note = Short ssDNA molecule
ctagcgcccgctcctttcgctttcttcccttcctttctcgccacgttcgc

SEQ ID NO: 120 - DNA, 50 bp, synthetic construct
  source 1..50: mol_type = other DNA; note = Short ssDNA molecule
gggcaggtagccggatcaagcgtatgcagccgccgcattgcatcagccat

SEQ ID NO: 121 - DNA, 50 bp, synthetic construct
  source 1..50: mol_type = other DNA; note = Short ssDNA molecule
acgctcagtggaacgaaaactcacgttaagggattttggtcatgagatta

(19) United States
(12) Patent Application Publication
(10) Pub. No.: US 2025/0250598 A1
(43) Pub. Date: Aug. 7, 2025

(54) METHOD FOR PRODUCING DOUBLE STRANDED DNA

(71) Applicant: MOLIGO TECHNOLOGIES AB, Solna (SE)

(72) Inventors: Cosimo DUCANI, Solna (SE); Roger SALVATORI, Solna (SE)

(21) Appl. No.: 18/856,778

(22) PCT Filed: Apr. 19, 2023
(86) PCT No.: PCT/EP2023/060150

§ 371 Date: Oct. 14, 2024

(30) Foreign Application Priority Data
Apr. 19, 2022 (GB) 2205696.4

(51) Int. Cl. C12P 19/34; C12N 9/00; C12N 15/10; C12N 15/66; C12N 15/70; C40B 40/06

(52) U.S. Cl. CPC C12P 19/34; C12N 9/93; C12N 15/1031; C12N 15/66; C12N 15/70; C40B 40/06; C12Y 605/01001

(57) ABSTRACT

The present invention provides a method for producing double stranded DNA (dsDNA) molecules. In particular, the invention provides a method that utilises a plurality of single stranded DNA (ssDNA) molecules to form a dsDNA complex in which the ssDNA molecules are ligated to produce the dsDNA molecule.

### FIELD OF THE INVENTION

The present invention is in the field of synthetic biology and relates to a method for producing double stranded DNA (dsDNA) molecules, e.g. long dsDNA molecules, such as comprising at least about 1000 bp. In particular, the invention provides a method that utilises a plurality of single stranded DNA (ssDNA) molecules to form a dsDNA complex in which the ssDNA molecules are ligated to produce the dsDNA molecule. Some of the ssDNA molecules may be extended prior to their ligation. The ssDNA molecules used in the invention may be the product of an enzymatic synthesis, such as an enzyme-mediated rolling circle amplification (RCA) reaction. The invention also provides the use of a plurality of ssDNA molecules in a ligase-mediated reaction to produce a dsDNA molecule. Double stranded DNA molecules obtained by the method are also provided. A library of dsDNA molecules comprising a plurality of different (e.g. functionalised or modified) dsDNA molecules obtained by the method is also provided.

### BACKGROUND TO THE INVENTION

Synthetic biology is widely recognised as an important and rapidly developing scientific field. While synthetic biology is a multidisciplinary area of research, a key aspect relates to genetic engineering, including the synthesis of new genes and the modification of existing genes. Significant developments in the last two decades have resulted in gene synthesis becoming more affordable, which in turn has permitted the advance of many fields and the development of new applications.

Currently, gene synthesis (i.e. DNA synthesis) is based on the assembly of short adjacent oligonucleotides, usually synthesized by solid-phase synthesis, particularly phosphoramidite chemistry. Typically, these ssDNA segments are 60-80 nucleotides (nt) long and cover both strands of the DNA duplex to be produced. In order to reduce the quantity of oligonucleotides necessary to generate a desired sequence (e.g. gene sequence), each ssDNA segment is designed to possess an annealing sequence of 15-25 nucleotides to the ssDNA segment present in the opposite strand. A thermostable DNA polymerase then randomly extends the assembled oligonucleotides, generating products of varied lengths, which are subsequently used as a template for a PCR reaction driven by an excess of the outermost of the assembled oligonucleotides. This is known as Polymerase cycling assembly (PCA) or Assembly PCR.

Importantly, both the synthesis and the assembly aspects of the method described above present stark limitations to the accurate production of dsDNA, e.g. gene synthesis.

First and foremost, solid phase chemical synthesis (e.g. phosphoramidite synthesis) is inefficient and error-prone. Secondly, the number of errors increases with the length of the oligonucleotides, while the product yield decreases. The error rate in the production of polynucleotides by solid-phase synthesis is significantly higher than the error rate of polymerase enzymes observed in nature, and increases dramatically with the length of the polynucleotide, such that purities of only 70% are common in commercial polynucleotides of around 50 nucleotides in length. This error rate makes solid-phase synthesis methods unsuitable for the production of longer polynucleotides. For instance, solid-phase synthesis typically has an error rate of >0.5%, which means that the production of a ssDNA molecule containing 100 nucleotides would result in a mixture of ssDNA molecules in which more than 40% of the ssDNA molecules contain at least one error, i.e. the synthesis product would have a purity of less than 60%. Finally, the assembly of the ssDNA segments (oligos) to form the target gene is error prone, especially if there are multiple oligonucleotides with similar sequences, due at least in part because the typical overlap between opposite strands is short.

Thus, errors in the starting products are compounded by further errors in the assembly process and result in errors in the final assembly product, which need to be removed. Consequently, the efficient and accurate synthesis of long DNA sequences (e.g. genes) using current methods is very challenging, time-consuming, labour-intensive and expensive. This is due to the highly demanding post-synthesis quality control procedures that are needed to identify and select a product with the correct sequence or, more commonly, to perform sequencing/error correction/re-cloning cycles. Moreover, even with these post assembly processes, the production of molecules with the correct sequences cannot always be achieved, particularly for molecules with long and/or complex or repetitive sequences.

In view of the problems with existing methods, there is a desire for alternative methods of producing dsDNA molecules, particularly methods which are effective at generating long dsDNA molecules (e.g. of about 1 kb or longer) with high yields and purity.

### SUMMARY OF THE INVENTION

In work leading up to the present invention, the inventors have unexpectedly determined that long dsDNA molecules may be produced precisely and efficiently using ligase-mediated assembly of ssDNA molecules. In particular, the methods and uses provided herein do not involve PCR methods which commonly result in by-products, especially when long and complex sequences are desired. In particular, the inventors have found that long, pure ssDNA building blocks (e.g. each comprising 150-10000 nucleotides) can be used to accurately assemble stable dsDNA complexes, which can be joined efficiently to produce a desired dsDNA molecule.

Whilst not wishing to be bound by theory, it is thought that the purity of the starting material (e.g. enzymatically synthesised ssDNA molecules), combined with the length of the annealing sequences, e.g. at least 50 bp (which drastically reduces instances of errors due to mis-annealing events), leads to the accurate assembly of ultrapure dsDNA molecules (e.g. genes or gene fragments). Advantageously, this minimises (e.g. eliminates) the need for post assembly processes and enables the assembly of any sequence, independently of length and complexity. Unexpectedly, the Examples demonstrate that the dsDNA molecules can be produced in a one-pot reaction comprising both the synthesis and assembly of the ssDNA molecules into the desired dsDNA molecule. This is particularly advantageous as it further simplifies the methods.

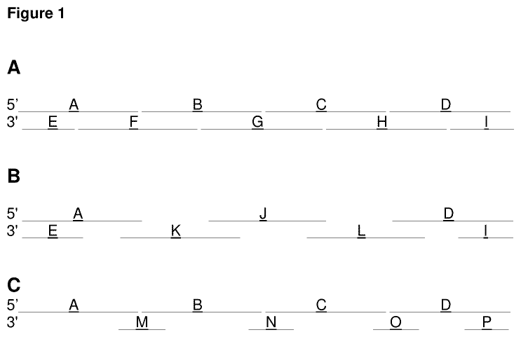

Representative strategies for assembling dsDNA complexes that can be joined to form the desired dsDNA molecules are shown in FIG. 1. These may be characterised as “complete overlapping” (FIG. 1A), “partial overlapping” (FIG. 1B) and “minimal overlapping” (FIG. 1C) strategies and are described below.

Complete overlapping: the plus strand of the target dsDNA (e.g. gene) is provided in four segments A, B, C and D (e.g. each comprising 1000 nucleotides); the minus strand is provided in five segments E, F, G, H and I (e.g. three segments each comprising 1000 nucleotides (F, G and H) and two segments each comprising 500nt (E and I). The smaller segments (E and I) are arranged to form the ends (3′ and 5′ ends, respectively) of the minus strand of the target dsDNA, so that the longer segments (F, G and H) comprise large regions of complementarity (overlap) with the segments in the plus strand. There are no gaps between the adjacent segments (i.e. their 5′ and 3′ ends are directly adjacent) such that the segments may be ligated in a ligase-mediated ligation reaction using the overlapping segment of the opposing strand to template the ligation reaction, i.e. segment A templates the ligation of segments E and F, segment F templates the ligation of segments A and B, and so on.

Partial overlapping: the plus strand of the target dsDNA (e.g. gene) is provided in three segments A, J and D (e.g. each comprising 1000 nucleotides), wherein segments A and D form the ends (5′ and 3′ ends, respectively) of the plus strand of the target dsDNA, and segment J comprises a central portion of the plus strand of the target dsDNA (e.g. it contains nucleotides 1501 to 2500 of the plus strand). The minus strand is provided in four segments E, K, L and I (e.g. two segments each comprising 1000 nucleotides (K and L) and two segments each comprising 500nt (E and I)). As with the complete overlapping method described above, the small segments, E and I, are arranged to form the ends (3′ and 5′ ends, respectively) of the minus strand of the target dsDNA. The segments are arranged such that there are gaps between adjacent segments (i.e. the 5′ and 3′ ends are indirectly adjacent) and wherein the longer segments only overlap partially, e.g. comprising regions of complementarity of about 250 bp. Formation of the DNA complex via hybridisation of complementary sequences allows the internal 3′ ends of the segments to be extended in a template-mediated polymerase extension reaction using the overlapping segment of the opposing strand to template the extension reaction, i.e. segment A templates the extension of segment K, segment K templates the extension of segment A, and so on. The extended segments then may be ligated in a ligase-mediated ligation reaction using the overlapping segment of the opposing strand to template the ligation reaction akin to the complete overlapping method, i.e. segment A templates the ligation of segments E and K, segment K templates the ligation of segments A and J, and so on.

Minimal overlapping: the plus strand is identical to the complete overlapping strategy and the segments A, B, C and D are used. The minus strand is provided by four smaller segments M, N, O and P (e.g. each containing 200 nucleotides). Only one of the segments provides an end of the minus strand, i.e. segment P provides the 5′ end of the minus strand. The other minus strand segments (M, N and O) overlap partially with the plus strand segments, e.g. comprising regions of complementarity of about 100 bp. Akin to the partial overlapping strategy described above, formation of the DNA complex via hybridisation of complementary sequences allows the internal 3′ ends of the minus strand segments to be extended in a template-mediated polymerase extension reaction using the overlapping segment of the plus strand to template the extension reaction, i.e. segment A templates the extension of segment M, segment B templates the extension of segment N, and so on. The extended segments then may be ligated in a ligase-mediated ligation reaction using the overlapping segment of the opposing strand to template the ligation reaction akin to the complete overlapping method, i.e. segment B templates the ligation of segments M and N, segment M templates the ligation of segments A and B, and so on.

It will be understood by the skilled person that the strategies outlined above may be adapted to increase the number of segments, e.g. to produce longer dsDNA molecules. Similarly, the lengths of the segments may be modified. For instance, it is not necessary that all of the segments of the plus and/or minus strand are the same length. In particular, the segments may be designed to ensure that the locations of the junctions (i.e. the positions in the dsDNA complex in which the 5′ and 3′ ends of the segments are directly adjacent) do not coincide with repetitive sequences and/or sequences capable of forming secondary structures, in order to minimise mis-alignments within the DNA complexes.

It will also be evident that a plurality of versions (e.g. sequence variants) of one or more of the ssDNA segments may be provided in order to generate a mixture of dsDNA molecules, i.e. to produce a gene library, such as for use in phage display methods etc. By way of example, and with reference to the strategies mentioned above and FIG. 1, several versions (e.g. sequence variants) of segment K could be provided in the reaction mixture which have common regions of complementarity and different sequences in the region opposite the gap in the opposing strand (i.e. the gap between segments A and J). As shown in Example 4 a library of ssDNA variants may be produced by including Manganese ions in the DNA synthesis reaction. The product of the extension and ligation reactions would be a mixture of dsDNA molecules comprising different sequences in the region provided by (and encoded by) the plurality of versions of segment K. Notably, the different versions of the ssDNA segments would not need to be sequence variants. For instance, a plurality of ssDNA molecules each comprising modified nucleotides (e.g. functionalised, such as methylated, nucleotides) could be provided, as shown in Example 5. The different versions may contain modifications at different positions and/or different modifications.

Moreover, it will be understood that one or more of the plurality of ssDNA molecules may comprise one or more modified nucleotides (e.g. functionalised, such as methylated, nucleotides), e.g. site-specific modifications, such that the modified nucleotides are incorporated in the dsDNA molecule produced. For instance, one or more of the ssDNA molecules may contain one or more methylated cytosines to produce a methylated dsDNA molecule (see Example 5), which may find particular utility in gene-silencing and epigenetic techniques.

It will be evident that the partial overlapping strategy described above may be modified such that the segment providing the 3′ end of the minus strand (segment E) may be omitted as the segment K may be extended using segment A as a template to provide the 3′ end of the minus strand of the dsDNA molecule. In other words, the segment providing the 3′ end of the minus (e.g. second) strand in the partial overlapping strategy is optional, akin to the minimal overlapping strategy.

Similarly, the segment providing the 3′ end of the plus strand (segment D) may be truncated, such that it does not contain the 3′ end of the plus strand of the target dsDNA, as segment D may be extended using segment I as a template to provide the 3′ end of the plus strand. Thus, in the partial overlapping strategy a segment of the plus strand and/or a segment of the minus strand may provide the 3′ end of the target dsDNA molecule via a templated extension reaction, i.e. a polymerase-mediated extension reaction.

Thus, provided herein is the use of a plurality of single stranded DNA (ssDNA) molecules in a ligase-mediated ligation reaction to produce a double stranded DNA (dsDNA) molecule comprising at least 1500 bp, wherein:
(i) each of a first plurality of single stranded DNA (ssDNA) molecules comprises (e.g. consists of) a non-overlapping portion of a first strand of the dsDNA molecule, wherein a first ssDNA molecule comprises the 5′ end of the first strand of the dsDNA molecule and a second ssDNA: (a) comprises the 3′ end of the first strand of the dsDNA molecule or (b) is capable of providing (provides) the 3′ end of the first strand of the dsDNA molecule via a polymerase-mediated extension reaction using a ssDNA molecule comprising the 5′ end of the second strand as a template; and
(ii) each of a second plurality of ssDNA molecules comprises (e.g. consists of) a non-overlapping portion of a second strand of the dsDNA molecule, wherein a first ssDNA molecule comprises the 5′ end of the second strand of the dsDNA molecule and, optionally, a second ssDNA comprises the 3′ end of the second strand of the dsDNA molecule,
wherein:
(1) at least two of the plurality of ssDNA molecules from (ii) each comprise a first region that is fully complementary to an end region of a ssDNA molecule of (i) and a second region that is fully complementary to an end region of a different ssDNA molecule of (i);
(2) the ssDNA molecules of (i) to which the ssDNA molecules of (ii) are complementary form adjacent portions of the first strand of the dsDNA molecule;
(3) the regions that are complementary comprise at least 50 nucleotides;
(4) all of the ssDNA molecules comprise at least 150 nucleotides; and
(5) the ssDNA molecules are provided in equimolar amounts;
and wherein when the first and second pluralities of ssDNA molecules are contacted under conditions suitable for hybridisation of DNA molecules they form a dsDNA complex comprising all of the ssDNA molecules that can be ligated in a ligase-mediated reaction to produce the dsDNA molecule.

Thus, provided herein is the use of a plurality of single stranded DNA (ssDNA) molecules in a ligase-mediated ligation reaction to produce a double stranded DNA (dsDNA) molecule comprising at least 1500 bp, wherein:
(i) each of a first plurality of single stranded DNA (ssDNA) molecules comprises (e.g. consists of) a non-overlapping portion of a first strand of the dsDNA molecule, wherein a first ssDNA molecule comprises the 5′ end of the first strand of the dsDNA molecule and a second ssDNA comprises the 3′ end of the first strand of the dsDNA molecule; and
(ii) each of a second plurality of ssDNA molecules comprises (e.g. consists of) a non-overlapping portion of a second strand of the dsDNA molecule, wherein a first ssDNA molecule comprises the 5′ end of the second strand of the dsDNA molecule and, optionally, a second ssDNA comprises the 3′ end of the second strand of the dsDNA molecule,
wherein:
(1) at least two of the plurality of ssDNA molecules from (ii) each comprise a first region that is fully complementary to an end region of a ssDNA molecule of (i) and a second region that is fully complementary to an end region of a different ssDNA molecule of (i);
(2) the ssDNA molecules of (i) to which the ssDNA molecules of (ii) are complementary form adjacent portions of the first strand of the dsDNA molecule;
(3) the regions that are complementary comprise at least 50 nucleotides;
(4) all of the ssDNA molecules comprise at least 150 nucleotides; and
(5) the ssDNA molecules are provided in equimolar amounts;
and wherein when the first and second pluralities of ssDNA molecules are contacted under conditions suitable for hybridisation of DNA molecules they form a dsDNA complex comprising all of the ssDNA molecules that can be ligated in a ligase-mediated reaction to produce the dsDNA molecule.

Also, provided herein is the use of a plurality of single stranded DNA (ssDNA) molecules in a ligase-mediated ligation reaction to produce a double stranded DNA (dsDNA) molecule comprising at least 1500 bp, wherein:
(i) a first plurality of ssDNA molecules comprises at least three ssDNA molecules each comprising (e.g. consisting of) a non-overlapping portion of a first strand of the dsDNA molecule, wherein a first ssDNA molecule comprises the 5′ end of the first strand of the dsDNA molecule and a second ssDNA comprises the 3′ end of the first strand of the dsDNA molecule; and
(ii) a second plurality of ssDNA molecules comprises at least four ssDNA molecules each comprising (e.g. consisting of) a non-overlapping portion of a second strand of the dsDNA molecule, wherein: a first ssDNA molecule comprises the 5′ end of the second strand of the dsDNA molecule and is fully complementary a region of the second ssDNA of (i); and a second ssDNA molecule comprises the 3′ end of the second strand of the dsDNA molecule and is fully complementary a region of the first ssDNA molecule of (i),
wherein:
(1) at least two of the at least four ssDNA molecules from (ii) that do not comprise the 5′ and 3′ ends of the second strand of the dsDNA molecule each comprise a first region that is fully complementary to an end region of a ssDNA molecule of (i) and a second region that is fully complementary to an end region of a different ssDNA molecule of (i);
(2) the ssDNA molecules of (i) to which the ssDNA molecules of (ii) are complementary form directly adjacent portions of the first strand of the dsDNA molecule;
(3) the regions that are complementary comprise at least 50 nucleotides;
(4) all of the ssDNA molecules comprise at least 150 nucleotides; and
(5) the ssDNA molecules are provided in equimolar amounts;
and wherein when the first and second pluralities of ssDNA molecules are contacted under conditions suitable for hybridisation of DNA molecules they form a dsDNA complex comprising all of the ssDNA molecules that can be ligated in a ligase-mediated ligation reaction to produce the dsDNA molecule.

Further provided herein is the use of a plurality of single stranded DNA (ssDNA) molecules in a ligase-mediated ligation reaction to produce a double stranded DNA (dsDNA) molecule comprising at least 1500 bp, wherein:
(i) a first plurality of ssDNA molecules comprises at least three ssDNA molecules each comprising (e.g. consisting of) a non-overlapping portion of a first strand of the dsDNA molecule, wherein: a first ssDNA molecule comprises the 5′ end of the first strand of the dsDNA molecule; and a second ssDNA molecule: (a) comprises the 3′ end of the first strand of the dsDNA molecule or (b) is capable of providing (provides) the 3′ end of the first strand of the dsDNA molecule via a polymerase-mediated extension reaction using a ssDNA molecule comprising the 5′ end of the second strand as a template; and
(ii) a second plurality of ssDNA molecules comprises at least three (e.g. at least four) ssDNA molecules each comprising (e.g. consisting of) a non-overlapping portion of a second strand of the dsDNA molecule, wherein: a first ssDNA molecule comprises the 5′ end of the second strand of the dsDNA molecule and is fully complementary a region of the second ssDNA of (i); and, optionally, a second ssDNA molecule comprises the 3′ end of the second strand of the dsDNA molecule and is fully complementary a region of the first ssDNA molecule of (i),
wherein:
(1) at least two of the at least three (e.g. at least two of the at least four) ssDNA molecules from (ii) that do not comprise the 5′ and 3′ ends of the second strand of the dsDNA molecule each comprise a first region that is fully complementary to an end region of a ssDNA molecule of (i) and a second region that is fully complementary to an end region of a different ssDNA molecule of (i);
(2) the ssDNA molecules of (i) to which the ssDNA molecules of (ii) are complementary form indirectly adjacent portions of the first strand of the dsDNA molecule and the ssDNA molecules of (ii) to which the ssDNA molecules of (i) are complementary form indirectly adjacent portions of the second strand of the dsDNA molecule;
(3) the regions that are complementary comprise at least 50 nucleotides;
(4) all of the ssDNA molecules comprise at least 150 nucleotides; and
(5) the ssDNA molecules are provided in equimolar amounts;
and wherein when the first and second pluralities of ssDNA molecules are contacted under conditions suitable for hybridisation of DNA molecules they form a partially dsDNA complex comprising all of the ssDNA molecules, wherein the internal 3′ ends of the ssDNA molecules in the partially dsDNA complex can be extended in a polymerase-mediated extension reaction to produce a fully dsDNA complex that can be ligated in a ligase-mediated ligation reaction to produce the dsDNA molecule.

Further provided herein is the use of a plurality of single stranded DNA (ssDNA) molecules in a ligase-mediated ligation reaction to produce a double stranded DNA (dsDNA) molecule comprising at least 1500 bp, wherein:
(i) a first plurality of ssDNA molecules comprises at least three ssDNA molecules each comprising (e.g. consisting of) a non-overlapping portion of a first strand of the dsDNA molecule, wherein: a first ssDNA molecule comprises the 5′ end of the first strand of the dsDNA molecule; and a second ssDNA molecule comprises the 3′ end of the first strand of the dsDNA molecule; and
(ii) a second plurality of ssDNA molecules comprises at least four ssDNA molecules each comprising (e.g. consisting of) a non-overlapping portion of a second strand of the dsDNA molecule, wherein: a first ssDNA molecule comprises the 5′ end of the second strand of the dsDNA molecule and is fully complementary a region of the second ssDNA of (i); and a second ssDNA molecule comprises the 3′ end of the second strand of the dsDNA molecule and is fully complementary a region of the first ssDNA molecule of (i),
wherein:
(1) at least two of the at least four ssDNA molecules from (ii) that do not comprise the 5′ and 3′ ends of the second strand of the dsDNA molecule each comprise a first region that is fully complementary to an end region of a ssDNA molecule of (i) and a second region that is fully complementary to an end region of a different ssDNA molecule of (i);
(2) the ssDNA molecules of (i) to which the ssDNA molecules of (ii) are complementary form indirectly adjacent portions of the first strand of the dsDNA molecule and the ssDNA molecules of (ii) to which the ssDNA molecules of (i) are complementary form indirectly adjacent portions of the second strand of the dsDNA molecule;
(3) the regions that are complementary comprise at least 50 nucleotides;
(4) all of the ssDNA molecules comprise at least 150 nucleotides; and
(5) the ssDNA molecules are provided in equimolar amounts;
and wherein when the first and second pluralities of ssDNA molecules are contacted under conditions suitable for hybridisation of DNA molecules they form a partially dsDNA complex comprising all of the ssDNA molecules, wherein the internal 3′ ends of the ssDNA molecules in the partially dsDNA complex can be extended in a polymerase-mediated extension reaction to produce a fully dsDNA complex that can be ligated in a ligase-mediated ligation reaction to produce the dsDNA molecule.

Also provided herein is the use of a plurality of single stranded DNA (ssDNA) molecules in a ligase-mediated ligation reaction to produce a double stranded DNA (dsDNA) molecule comprising at least 1500 bp, wherein:
(i) a first plurality of ssDNA molecules comprises at least three ssDNA molecules each comprising (e.g. consisting of) a non-overlapping portion of a first strand of the dsDNA molecule, wherein: a first ssDNA molecule comprises the 5′ end of the first strand of the dsDNA molecule; and a second ssDNA molecule comprises the 3′ end of the first strand of the dsDNA molecule; and
(ii) a second plurality of ssDNA molecules comprises at least three ssDNA molecules each comprising (e.g. consisting of) a non-overlapping portion of a second strand of the dsDNA molecule, wherein: a first ssDNA molecule comprises the 5′ end of the second strand of the dsDNA molecule and is fully complementary a region of the second ssDNA molecule of (i),
wherein:
(1) at least two of the at least three ssDNA molecules from (ii) that do not comprise the 5′ end of the second strand of the dsDNA molecule each comprise a first region that is fully complementary to an end region of a ssDNA molecule of (i) and a second region that is fully complementary to an end region of a different ssDNA molecule of (i);
(2) the ssDNA molecules of (i) to which the ssDNA molecules of (ii) are complementary form directly adjacent portions of the first strand of the dsDNA molecule and the ssDNA molecules of (ii) to which the ssDNA molecules of (i) are complementary form indirectly adjacent portions of the second strand of the dsDNA molecule;
(3) the regions that are complementary comprise at least 50 nucleotides;
(4) all of the ssDNA molecules comprise at least 150 nucleotides; and
(5) the ssDNA molecules are provided in equimolar amounts;
and wherein when the first and second pluralities of ssDNA molecules are contacted under conditions suitable for hybridisation of DNA molecules they form a partially dsDNA complex comprising all of the ssDNA molecules, wherein the internal 3′ ends of the ssDNA molecules in the partially dsDNA complex (the internal 3′ ends of the second plurality of ssDNA molecules) can be extended in a polymerase-mediated extension reaction to produce a fully dsDNA complex that can be ligated in a ligase-mediated ligation reaction to produce the dsDNA molecule.

Alternatively viewed, provided herein is a method for producing a double stranded DNA (dsDNA) molecule comprising at least 1500 bp, the method comprising:
(a) providing:
(i) a plurality of single stranded DNA (ssDNA) molecules each comprising (e.g. consisting of) a non-overlapping portion of a first strand of the dsDNA molecule, wherein a first ssDNA molecule comprises the 5′ end of the first strand of the dsDNA molecule and a second ssDNA: (a) comprises the 3′ end of the first strand of the dsDNA molecule or (b) is capable of providing (provides) the 3′ end of the first strand of the dsDNA molecule via a polymerase-mediated extension reaction using a ssDNA molecule comprising the 5′ end of the second strand as a template; and
(ii) a plurality of ssDNA molecules each comprising (e.g. consisting of) a non-overlapping portion of a second strand of the dsDNA molecule, wherein a first ssDNA molecule comprises the 5′ end of the second strand of the dsDNA molecule and, optionally, a second ssDNA comprises the 3′ end of the second strand of the dsDNA molecule,
wherein:
(1) at least two of the plurality of ssDNA molecules from (ii) each comprise a first region that is fully complementary to an end region of a ssDNA molecule of (i) and a second region that is fully complementary to an end region of a different ssDNA molecule of (i);
(2) the ssDNA molecules of (i) to which the ssDNA molecules of (ii) are complementary form adjacent portions of the first strand of the dsDNA molecule;
(3) the regions that are complementary comprise at least 50 nucleotides;
(4) all of the ssDNA molecules comprise at least 150 nucleotides; and
(5) the ssDNA molecules are provided in equimolar amounts;
(b) hybridizing the ssDNA molecules that contain regions of complementarity to produce a dsDNA complex comprising all of the ssDNA molecules from (a); and
(c) ligating adjacent ssDNA molecules to produce the dsDNA molecule.

Also provided herein is a method for producing a double stranded DNA (dsDNA) molecule comprising at least 1500 bp, the method comprising:
(a) providing:
(i) a plurality of single stranded DNA (ssDNA) molecules each comprising (e.g. consisting of) a non-overlapping portion of a first strand of the dsDNA molecule, wherein a first ssDNA molecule comprises the 5′ end of the first strand of the dsDNA molecule and a second ssDNA comprises the 3′ end of the first strand of the dsDNA molecule; and
(ii) a plurality of ssDNA molecules each comprising (e.g. consisting of) a non-overlapping portion of a second strand of the dsDNA molecule, wherein a first ssDNA molecule comprises the 5′ end of the second strand of the dsDNA molecule and, optionally, a second ssDNA comprises the 3′ end of the second strand of the dsDNA molecule,
wherein:
(1) at least two of the plurality of ssDNA molecules from (ii) each comprise a first region that is fully complementary to an end region of a ssDNA molecule of (i) and a second region that is fully complementary to an end region of a different ssDNA molecule of (i);
(2) the ssDNA molecules of (i) to which the ssDNA molecules of (ii) are complementary form adjacent portions of the first strand of the dsDNA molecule;
(3) the regions that are complementary comprise at least 50 nucleotides;
(4) all of the ssDNA molecules comprise at least 150 nucleotides; and
(5) the ssDNA molecules are provided in equimolar amounts;
(b) hybridizing the ssDNA molecules that contain regions of complementarity to produce a dsDNA complex comprising all of the ssDNA molecules from (a); and
(c) ligating adjacent ssDNA molecules to produce the dsDNA molecule.

Also provided herein is a method for producing a double stranded DNA (dsDNA) molecule comprising at least 1500 bp, the method comprising:
(a) providing:
(i) at least three ssDNA molecules each comprising (e.g. consisting of) a non-overlapping portion of a first strand of the dsDNA molecule, wherein a first ssDNA molecule comprises the 5′ end of the first strand of the dsDNA molecule and a second ssDNA comprises the 3′ end of the first strand of the dsDNA molecule; and
(ii) at least four ssDNA molecules each comprising (e.g. consisting of) a non-overlapping portion of a second strand of the dsDNA molecule, wherein: a first ssDNA molecule comprises the 5′ end of the second strand of the dsDNA molecule and is fully complementary a region of the second ssDNA of (i); and a second ssDNA molecule comprises the 3′ end of the second strand of the dsDNA molecule and is fully complementary a region of the first ssDNA molecule of (i),
wherein:
(1) at least two of the at least four ssDNA molecules from (ii) that do not comprise the 5′ and 3′ ends of the second strand of the dsDNA molecule each comprise a first region that is fully complementary to an end region of a ssDNA molecule of (i) and a second region that is fully complementary to an end region of a different ssDNA molecule of (i);
(2) the ssDNA molecules of (i) to which the ssDNA molecules of (ii) are complementary form directly adjacent portions of the first strand of the dsDNA molecule;
(3) the regions that are complementary comprise at least 50 nucleotides;
(4) all of the ssDNA molecules comprise at least 150 nucleotides; and
(5) the ssDNA molecules are provided in equimolar amounts;
(b) hybridizing the ssDNA molecules that contain regions of complementarity to produce a fully dsDNA complex comprising all of the ssDNA molecules from (a); and
(c) ligating directly adjacent ssDNA molecules to produce the dsDNA molecule.

Further provided herein is a method for producing a double stranded DNA (dsDNA) molecule comprising at least 1500 bp, the method comprising:
(a) providing:
(i) at least three ssDNA molecules each comprising (e.g. consisting of) a non-overlapping portion of a first strand of the dsDNA molecule, wherein: a first ssDNA molecule comprises the 5′ end of the first strand of the dsDNA molecule; and a second ssDNA molecule: (a) comprises the 3′ end of the first strand of the dsDNA molecule or (b) is capable of providing (provides) the 3′ end of the first strand of the dsDNA molecule via a polymerase-mediated extension reaction using a ssDNA molecule comprising the 5′ end of the second strand as a template; and
(ii) at least three (e.g. at least four) ssDNA molecules each comprising (e.g. consisting of) a non-overlapping portion of a second strand of the dsDNA molecule, wherein: a first ssDNA molecule comprises the 5′ end of the second strand of the dsDNA molecule and is fully complementary a region of the second ssDNA of (i); and, optionally, a second ssDNA molecule comprises the 3′ end of the second strand of the dsDNA molecule and is fully complementary a region of the first ssDNA molecule of (i),
wherein:
(1) at least two of the at least three (e.g. at least two of the at least four) ssDNA molecules from (ii) that do not comprise the 5′ and 3′ ends of the second strand of the dsDNA molecule each comprise a first region that is fully complementary to an end region of a ssDNA molecule of (i) and a second region that is fully complementary to an end region of a different ssDNA molecule of (i);
(2) the ssDNA molecules of (i) to which the ssDNA molecules of (ii) are complementary form indirectly adjacent portions of the first strand of the dsDNA molecule and the ssDNA molecules of (ii) to which the ssDNA molecules of (i) are complementary form indirectly adjacent portions of the second strand of the dsDNA molecule;
(3) the regions that are complementary comprise at least 50 nucleotides;
(4) all of the ssDNA molecules comprise at least 150 nucleotides; and
(5) the ssDNA molecules are provided in equimolar amounts;
(b) hybridizing the ssDNA molecules that contain regions of complementarity to produce a partially dsDNA complex comprising all of the ssDNA molecules from (a);
(c) extending the 3′ ends of the ssDNA molecules to produce a fully dsDNA complex; and
(d) ligating directly adjacent ssDNA molecules to produce the dsDNA molecule.

Further provided herein is a method for producing a double stranded DNA (dsDNA) molecule comprising at least 1500 bp, the method comprising:
(a) providing:
(i) at least three ssDNA molecules each comprising (e.g. consisting of) a non-overlapping portion of a first strand of the dsDNA molecule, wherein: a first ssDNA molecule comprises the 5′ end of the first strand of the dsDNA molecule; and a second ssDNA molecule comprises the 3′ end of the first strand of the dsDNA molecule; and
(ii) at least four ssDNA molecules each comprising (e.g. consisting of) a non-overlapping portion of a second strand of the dsDNA molecule, wherein: a first ssDNA molecule comprises the 5′ end of the second strand of the dsDNA molecule and is fully complementary a region of the second ssDNA of (i); and a second ssDNA molecule comprises the 3′ end of the second strand of the dsDNA molecule and is fully complementary a region of the first ssDNA molecule of (i),
wherein:
(1) at least two of the at least four ssDNA molecules from (ii) that do not comprise the 5′ and 3′ ends of the second strand of the dsDNA molecule each comprise a first region that is fully complementary to an end region of a ssDNA molecule of (i) and a second region that is fully complementary to an end region of a different ssDNA molecule of (i);
(2) the ssDNA molecules of (i) to which the ssDNA molecules of (ii) are complementary form indirectly adjacent portions of the first strand of the dsDNA molecule and the ssDNA molecules of (ii) to which the ssDNA molecules of (i) are complementary form indirectly adjacent portions of the second strand of the dsDNA molecule;
(3) the regions that are complementary comprise at least 50 nucleotides;
(4) all of the ssDNA molecules comprise at least 150 nucleotides; and
(5) the ssDNA molecules are provided in equimolar amounts;
(b) hybridizing the ssDNA molecules that contain regions of complementarity to produce a partially dsDNA complex comprising all of the ssDNA molecules from (a);
(c) extending the 3′ ends of the ssDNA molecules to produce a fully dsDNA complex; and
(d) ligating directly adjacent ssDNA molecules to produce the dsDNA molecule.

Yet further provided herein is a method for producing a double stranded DNA (dsDNA) molecule comprising at least 1500 bp, the method comprising:
(a) providing:
(i) at least three ssDNA molecules each comprising (e.g. consisting of) a non-overlapping portion of a first strand of the dsDNA molecule, wherein: a first ssDNA molecule comprises the 5′ end of the first strand of the dsDNA molecule; and a second ssDNA molecule comprises the 3′ end of the first strand of the dsDNA molecule; and
(ii) at least three ssDNA molecules each comprising (e.g. consisting of) a non-overlapping portion of a second strand of the dsDNA molecule, wherein: a first ssDNA molecule comprises the 5′ end of the second strand of the dsDNA molecule and is fully complementary a region of the second ssDNA molecule of (i),
wherein:
(1) at least two of the at least three ssDNA molecules from (ii) that do not comprise the 5′ end of the second strand of the dsDNA molecule each comprise a first region that is fully complementary to an end region of a ssDNA molecule of (i) and a second region that is fully complementary to an end region of a different ssDNA molecule of (i);
(2) the ssDNA molecules of (i) to which the ssDNA molecules of (ii) are complementary form directly adjacent portions of the first strand of the dsDNA molecule and the ssDNA molecules of (ii) to which the ssDNA molecules of (i) are complementary form indirectly adjacent portions of the second strand of the dsDNA molecule;
(3) the regions that are complementary comprise at least 50 nucleotides;
(4) all of the ssDNA molecules comprise at least 150 nucleotides; and
(5) the ssDNA molecules are provided in equimolar amounts;
(b) hybridizing the ssDNA molecules that contain regions of complementarity to produce a partially dsDNA complex comprising all of the ssDNA molecules from (a);
(c) extending the 3′ ends of the ssDNA molecules of (ii) to produce a fully dsDNA complex; and
(d) ligating directly adjacent ssDNA molecules to produce the dsDNA molecule.

While the methods and uses provided herein find particular utility in the production (i.e. synthesis) of long dsDNA molecules (e.g. comprising at least 1500 bp), it will be understood by the skilled person that the method and uses provided herein also may be particularly advantageous for the production of dsDNA molecules that contain repetitive sequences and/or sequences capable of forming secondary structures. In view of the challenges associated with the production of even small dsDNA molecules that contain repetitive sequences and/or sequences capable of forming secondary structures, when the methods and uses provided herein are applied to the production of dsDNA molecules that contain repetitive sequences and/or sequences capable of forming secondary structures, the size requirements set out above need not apply. Thus, for instance, the methods and uses provided herein may be used to produce dsDNA molecules comprising 500 bp or more (e.g. 750 bp or 1000 bp or more), when the dsDNA molecules contain repetitive sequences and/or sequences capable of forming secondary structures.

Also provided herein are dsDNA molecules produced by the methods and uses provided herein.

Further provided herein is a library of dsDNA molecules comprising a plurality of different (e.g. functionalised or modified) dsDNA molecules obtained by the methods and uses provided herein.

### DETAILED DESCRIPTION

The terms “double stranded DNA (dsDNA)” and “dsDNA molecule” are used interchangeably herein and refer to nucleic acid molecules composed of two anti-parallel polynucleotides comprising deoxyribonucleotides (dNTPs) held together by hydrogen bonding via Watson-Crick type or analogous base pair interactions. The two anti-parallel polynucleotides of a dsDNA molecule may be termed DNA “strands” and may be distinguished as “first and second strands” or “plus and minus strands”.

In view of the fact that synthetic and/or functionalised nucleotides (i.e. modified nucleotides) are capable of participating in Watson-Crick type or analogous base pair interactions, it will be evident that a dsDNA does not need to be completely composed of standard or conventional dNTPs, i.e. dsDNA may comprise synthetic and/or functionalised nucleotides. For instance, 5′ and/or 3′ nucleotides in a dsDNA molecule may be modified, e.g. post-synthesis. Moreover, synthetic and/or functionalised nucleotides may be incorporated in DNA molecules during their synthesis, e.g. during the synthesis of single stranded DNA (ssDNA) molecules used to produce a double stranded molecule described herein. Thus, it is contemplated that dsDNA produced according to the methods and uses provided herein may contain one or more functionalised, modified or non-standard nucleotides, e.g. methylated nucleotides, such as methylated cytosine. Thus, the dsDNA molecule provided by the methods and uses described herein may be a modified (e.g. functionalised) dsDNA molecule, e.g. a methylated dsDNA molecule. In particular, the dsDNA molecule provided by the methods and uses described herein may contain one or more (e.g. a plurality of) site-specific modifications. For instance, the dsDNA molecule may contain one or more site-specific modifications relative to the corresponding native dsDNA molecule. However, the majority of the nucleotides in the dsDNA molecules will be conventional dNTPs, e.g. at least 80, 85, 90%, 95%, 96%, 97%, 98% or 99% of the nucleotides in the dsDNA molecules will be conventional dNTPs. Thus, the dsDNA molecule produced according to the methods and uses provided herein may consist of conventional dNTPs.

Alternatively viewed, one or more of the plurality of ssDNA molecules provided for use in the method and use may comprise one or more modified nucleotides (e.g. functionalised, such as methylated, nucleotides), e.g. site-specific modifications, i.e. such that the modified nucleotides are incorporated in the dsDNA molecule produced.

The terms “conventional nucleotides” and “standard nucleotides” are used interchangeably herein and refer to deoxynucleotides comprising one of the four bases found in DNA; adenine, guanine, cytosine and thymine. The terms “conventional nucleotides” thus encompasses, for example, dATP, dGTP, dCTP and dTTP. Whilst uracil is not typically found in DNA naturally, dUTP readily may be used instead of, or in addition to, dTTP. Thus, in the context of the present invention, dUTP may be viewed as a “conventional” nucleotide.

The terms “functionalised nucleotides” or “functionalised dNTPs” refer to nucleotides that comprise a modification relative to unmodified conventional nucleotides, wherein said modification provides said functionalised nucleotides and/or polynucleotides comprising at least one functionalised nucleotide with additional or alternative properties or characteristics, i.e. relative to the corresponding conventional nucleotide. Thus, functionalised nucleotides may also be referred to as synthetic, modified or non-standard nucleotides. For instance, the modification may render the nucleotide detectable, e.g. by the incorporation of a label, or capable of interacting and/or reacting with another component, i.e. a component with which the corresponding conventional nucleotide does not interact or react. The modification may render the polynucleotide containing the nucleotide resistant to degradation, e.g. chemical and/or enzymatic degradation (e.g. nuclease degradation), or may alter the metabolism of the nucleotide. The modification may be a modification that occurs naturally in vivo, such as methylation (e.g. the modified nucleotide may be 5-methylcytosine). Examples of functionalised nucleotides are provided in WO2020/161187, which is incorporated herein by reference.

The two anti-parallel polynucleotides hybridise to form a dsDNA molecule because they contain nucleotide sequences that are sufficiently complementary to form duplexes via Watson-Crick base pairing or analogous base pair interactions. Two nucleotide sequences are “complementary” to one another when those molecules share base pair organization homology. “Complementary” nucleotide sequences will combine with specificity to form a stable duplex under appropriate hybridization conditions. For instance, two sequences are complementary when a section of a first sequence can bind (hybridise) to a section of a second sequence in an anti-parallel sense wherein the 3′-end of each sequence binds to the 5′-end of the other sequence and each A, T (U), G and C of one sequence is then aligned with a T (U), A, C and G, respectively, of the other sequence. Thus, two sequences need not have perfect homology to be “complementary”. Polynucleotides comprising sequences that do not have perfect homology may be described as having partial complementarity (i.e. as being partially complementary) and hybridise to form a partially dsDNA molecule, i.e. comprising regions of mismatched base pairs that result in regions of the molecule that are single stranded. Thus, polynucleotides comprising sequences that have perfect homology may be described has having full complementarity (i.e. as being fully complementary) and hybridise to form a fully dsDNA molecule, i.e. comprising no regions of mismatched base pairs.

It will be understood that a partially dsDNA molecule may also refer to a DNA molecule in which there are no mismatched base pairs, i.e. a molecule comprising regions of dsDNA and regions of ssDNA where there is no opposing strand (such that there is a gap in one of the DNA strands). Such molecules may be referred to herein as partially dsDNA complexes. As noted above, strategies that result in partially dsDNA complexes may be used to generate mixtures of dsDNA molecules (e.g. libraries of dsDNA molecules, such as gene libraries) by using a plurality of ssDNA molecules with common regions of complementarity that border (i.e. flank) intervening variant sequences.

A dsDNA molecule produced according to the methods and uses provided herein may be fully complementary, i.e. fully double stranded, and this is preferred. However, it is contemplated that it may be desirable in some circumstances to produce dsDNA molecules that are partially complementary, e.g. when at least about 90% (and most preferably at least about 95%, 96%, 97%, 98% or 99%) of the nucleotides share base pair organization over the full length of the molecule. Thus, for instance, a dsDNA molecule may have regions that are fully complementary separated by one or more regions with partial or no complementarity, such that overall at least about 90% of the nucleotides form a base pair with the complementary nucleotides on the opposing strand.

The method or use provided herein finds particular utility in the production of long dsDNA molecules. A long dsDNA refers to any length of dsDNA molecule that cannot be synthesised efficiently and/or accurately using conventional methods described above, such as PCA. As the efficiency and accuracy of a synthesis reaction may differ based on the complexity of the dsDNA to be produced, a long dsDNA molecule may include dsDNA molecules that are less than 1 kbp in length, e.g. 500-1000 bp, wherein the molecule cannot be synthesised efficiently and/or accurately using conventional methods, such as PCA. For instance, the method or use provided herein may find utility in synthesising any dsDNA molecules (irrespective of length) than cannot be produced by conventional methods, such as PCR assembly methods, with more than 70% accuracy, i.e, wherein 30% or more of the synthesis products contain an incorrect sequence.

Preferably, a dsDNA molecule produced by the method or use provided herein may comprise at least about 1500 bp (base pairs), such as at least about 1750 bp, 2000 bp, 2250 bp, 2500 bp, 3000 bp, 3500 bp or 4000 bp. A dsDNA molecule produced by the method or use provided herein may be very long, e.g. it may comprise 5 kbp, 6 kbp, 7 kbp, 8 kbp, 9 kbp, 10 kbp or more, such as 15 kbp, 20 kbp, 25 kbp, 30 kbp, 35 kbp, 40 kbp or more. For instance, the dsDNA may contain about 1-50 kbp, such as about 1.5-50 kbp, 2-45 kbp, 3-40 kbp, 4-35 kbp or 5-30 kbp.

The method and use provided herein advantageously may be used to produce dsDNA molecules comprising any sequence. The dsDNA molecule may comprise a coding sequence (i.e. RNA coding sequence) and/or a non-coding sequence (e.g. promoter). The coding sequence may encode any cellular or viral RNA. Thus, it may be mRNA, tRNA, rRNA, viral RNA, small nuclear RNA (snRNA), small nucleolar RNA (snoRNA), microRNA (miRNA), small interfering RNA (siRNA), piwi-interacting RNA (piRNA), ribozymal RNA, antisense RNA or non-coding RNA (e.g. a long non-coding RNA). Thus, the dsDNA may encode mRNA and may therefore be viewed as comprising a polypeptide encoding sequence. The non-coding sequence may be any non-coding DNA, such as an expression control sequence (e.g. a promoter, enhancer, terminator or functional portion thereof) or a recombinant DNA cloning vehicle or vector. For instance, the dsDNA may comprise a non-coding sequence (e.g. promoter) operatively (operably) linked to a coding sequence (e.g. polypeptide encoding sequence). Thus, the dsDNA may comprise a gene. It will be understood that the dsDNA may comprise any combination of coding and non-coding sequences, including combinations that do not occur in nature, e.g. a promoter sequence operatively linked to a heterologous polypeptide encoding sequence. The dsDNA produced by the method and use provided herein may be viewed as the “target dsDNA” or “desired dsDNA” and these terms are used interchangeably herein.

The method and use provided herein may be particularly beneficial for the production of dsDNA molecules that contain repetitive sequences and/or sequences that are capable of forming secondary structures. In this respect, the formation of dsDNA complexes using single stranded polynucleotides (e.g. ssDNA) that contain repetitive sequences and/or sequences that are capable of forming secondary structures typically results in a mixture of different complexes (both intermolecular and intramolecular complexes), as each polynucleotide may have complementarity to more than polynucleotide in the mixture. The formation of dsDNA molecules from these dsDNA complexes using conventional methods (e.g. using PCA) therefore results in a mixture of different dsDNA molecules, only some of which may contain the desired sequence. However, it is hypothesised that the use of single stranded polynucleotides that contain long regions of complementarity, e.g. at least 50 bp, may drive the formation of dsDNA complexes comprising the desired (i.e. correct) sequence, such that these complexes are present in excess. Moreover, the formation of dsDNA molecules from these complexes using ligase-mediated ligation reactions (optionally preceded by polymerase-mediated extension reactions), rather than PCR based methods (e.g. PCA), is thought to improve the yield of dsDNA molecules with the desired sequence. Thus, the method or use provided herein may be used for producing a dsDNA molecule comprising a repetitive sequence and/or a sequence that is capable of forming a secondary structure, optionally wherein the dsDNA comprises at least about 500 bp, e.g. at least about 750 bp, 1000 bp, 1250 bp or 1500 bp.

Accordingly, the method or use provided herein does not involve the polymerase chain reaction (PCR), e.g. PCA or assembly PCR, to produce the dsDNA. Alternatively viewed, the step of forming or producing the dsDNA molecule does not use the polymerase chain reaction (PCR), e.g. PCA or assembly PCR. As noted above, the step of producing the dsDNA molecule from the dsDNA complex involves a ligase-mediated ligation reaction, optionally preceded by a polymerase-mediated extension reaction. Notably, the ssDNA molecules used in the method provided herein may be produced using a method that involves PCR, e.g. asymmetric PCR. Thus, it will be understood that the step of forming the dsDNA molecule does not involve PCR.

A repetitive or repeat sequence refers to a nucleotide sequence that is present in a polynucleotide more than once. For instance, a region of a polynucleotide (e.g. 2 or more nucleotides, typically 3-10 nucleotides or more, such as 10-60 nucleotides) containing a single type of nucleotide (e.g. dATP) may be viewed as a repeat. Similarly, a more complex sequence (i.e. comprising more than one type of nucleotide) that is repeated one or more times in a polynucleotide may be viewed as a repeat. Repeated sequences may be tandem (i.e. directly adjacent to each other) or interspersed (i.e. with a non-repeating sequence separating the repeats). Thus, a repetitive sequence may comprise a sequence of at least 3 nucleotides (e.g. at least 4, 5 or 6 nucleotides, such as about 10, 15 or 20 nucleotides) that is present in a polynucleotide more than once, e.g. at least 2, 3, 4 or more times, such as 5, 6, 7, 8, 9, 10 or more times.

A sequence that is capable of forming a secondary structure refers to a nucleotide sequence that has regions of self-complementarity (full or partial complementarity) such that a polynucleotide comprising the sequence that is subjected to conditions suitable to allow hybridisation of complementary sequences forms one or more secondary structures, e.g. one or more intramolecular double stranded regions. The intramolecular double stranded regions may be separated by single stranded regions, such that the polynucleotide contains one or more so-called stem-loop regions.

The method and use provided herein use a plurality of single stranded DNA (ssDNA) molecules (polynucleotides) to form each strand of the dsDNA molecule. Each ssDNA molecule therefore provides a “non-overlapping portion” of a strand of the dsDNA molecule. Thus, the term “non-overlapping portion” means that each ssDNA molecule provides a distinct (discrete or different) portion (fragment, segment etc.) of one strand of the dsDNA, which is not contained in any of the other ssDNA molecules. While more than one ssDNA that provides a portion of one strand of the dsDNA molecule may contain the same sequence, these sequences are found in different parts of the strand of the dsDNA molecule, e.g. due to repetition of the sequence in the strand of the dsDNA molecule. Thus, when compared across their full-length, each ssDNA molecule of a plurality of ssDNA molecules has a different nucleotide sequence.

Thus, when the plurality of ssDNA molecules provided by the method and use are aligned end-to-end in the correct order, they form at least part of each stand of the dsDNA molecule, including at least the 5′ ends of the first and second strands, preferably the 5′ and 3′ ends of a first strand and at least the 5′ end of the second strand.

As noted above, each ssDNA molecule used in the methods and uses provided herein may be provided in a plurality of versions, i.e. different versions (e.g. sequence variants) of the equivalent non-overlapping portion of a strand of the dsDNA molecule, such that the product of the ligase-mediated ligation reaction is a mixture of dsDNA molecules. The dsDNA molecules in the mixture differ based on which version of the ssDNA molecule that was incorporated in the dsDNA complex. If the different versions of the ssDNA molecules differ by virtue of functional groups on the nucleotides (e.g. the position and/or type of functional group), the sequences of the different versions may be identical.

The term “sequence variant” refers to nucleotide sequences that share some sequence identity, but are not 100% identical. Thus, in the context of the present methods, sequence variants are ssDNA molecules that contain sequences that are identical (i.e. the sequences that form the regions of complementarity), which border (flank) intervening sequences that are not identical. The intervening sequences may be similar to each other (e.g. all of the intervening sequences in the plurality may comprise at least 60%, 70%, 80%, 90% or more sequence identity to each other, e.g. 95%, 96%, 97%, 98% or 99% sequence identity to each other) or divergent from each other (e.g. all of the intervening sequences in the plurality may comprise less than 60%, 50% or 40% sequence identity, e.g. 30%, 25%, 20%, or less sequence identity). It will be understood that the sequence identity values above may apply to the whole sequences also taking into account the identical end sequences. Sequence identity may be determined by any appropriate method known in the art, e.g. the using BLAST alignment algorithm.

The term “different” in the context of different non-overlapping portions of the stand of the dsDNA molecule refers to ssDNA molecules comprising nucleotide sequences in which one or more different nucleotides. Thus, different ssDNA molecules may comprise nucleotides sequences that differ by one or more, e.g. 2, 3, 4, 5, 6, 7, 8, 9, 10 or more, nucleotides, e.g. 20, 30, 40, 50, 60, 70, 80, 90 or more nucleotides. The differences may be in the length and/or sequence of the ssDNA molecules. Alternatively viewed, the different ssDNA molecules have less than 100% sequence identity each other, such as less than 99%, 98%, 97%, 96%, 95%, 90%, 85%, 80%, 70%, 60%, 50% to each other.

For instance, in the “complete overlapping” strategy described above, alignment of the non-overlapping ssDNA molecules end-to-end provides the complete sequence of both strands of the dsDNA, i.e. there are no “gaps” in the sequence. Thus, the ssDNA molecules used in the “complete overlapping” strategy may be viewed as consecutive ssDNA molecules.

In the “partial overlapping” strategy described above, alignment of the non-overlapping ssDNA molecules end-to-end provides the incomplete sequence of both strands of the dsDNA, i.e. there are “gaps” in the sequences of both strands. Thus, the ssDNA molecules provided in the “partial overlapping” strategy may be viewed as non-consecutive ssDNA molecules.

The term “gaps” as used herein refers to parts of the nucleotide sequence of a strand of the dsDNA molecule that are not provided directly by the plurality of ssDNA molecules. As discussed in more detail below, the gaps are “filled-in” using the sequence of the opposing strand as a template for a polymerase-mediated extension reaction. Thus, it may be considered that these parts of the nucleotide sequence of a strand of the dsDNA molecule are provided indirectly. For instance, in the “partial overlapping” strategy described above, the 3′ ends of the first and/or second strands of the dsDNA molecule may be provided indirectly.

In the “minimal overlapping” strategy described above, alignment of the non-overlapping ssDNA molecules end-to-end provides the complete sequence of a first strand of the dsDNA and the incomplete sequence of a second strand. Thus, the plurality of ssDNA molecules that provide the first strand in the “minimal overlapping” strategy may be viewed as consecutive ssDNA molecules and the plurality of ssDNA molecules that provide the second strand in the “minimal overlapping” strategy may be viewed as non-consecutive ssDNA molecules.

It will be understood that the strategies outlined above may be combined such that one or both stands may be provided by a mixture of consecutive and non-consecutive ssDNA molecules, such that only some of the ssDNA molecules need to be extended in a polymerase-mediated extension reaction. Thus, in some aspects, the plurality of ssDNA molecules that provide one strand of the dsDNA are a mixture of consecutive and non-consecutive ssDNA molecules and/or the plurality of ssDNA molecules that provide the other strand of the dsDNA are a mixture of consecutive and non-consecutive ssDNA molecules.

However, in some aspects the plurality of ssDNA molecules provided by the method and use are all consecutive ssDNA molecules. In some aspects the plurality of ssDNA molecules provided by the method and use are all non-consecutive ssDNA molecules. In some aspects, the plurality of ssDNA molecules that provide one strand of the dsDNA molecule are all consecutive ssDNA molecules and the plurality of ssDNA molecules that provide the other strand of the dsDNA molecule are all non-consecutive ssDNA molecules.

The ssDNA molecules (polynucleotides) may be any size suitable to produce a dsDNA molecule of the size described above and to provide a region of complementarity with its opposite strand of at least 50 base pairs (bp), as defined further below. It will be evident that the size of each ssDNA molecule in each plurality will depend on the number of molecules, and the strategy (e.g. complete overlapping, partial overlapping etc.), used to produce each strand of the dsDNA molecule. Typically, consecutive ssDNA molecules will be longer than non-consecutive ssDNA molecules. Accordingly, each ssDNA of a plurality of ssDNA molecules may be a different size. However, in some embodiments, each ssDNA molecule of a plurality of ssDNA molecules used to provide a strand of the dsDNA molecule may be substantially the same size, e.g. within about 20% (e.g. within about 15% or 10%) of the total number of nucleotides of each other, i.e. ±10% (e.g. ±7.5% or 5%) of the total number of nucleotides of each other. Thus, in a representative example, each ssDNA of a plurality of ssDNA molecules used to provide a strand of the dsDNA may consist of about 450-550 nucleotides (i.e. 500 nucleotides±50 nucleotides). However, it will be evident that the ssDNA molecules that provide the 5′ and 3′ ends of one of the strands of the dsDNA molecule (e.g. the second strand as defined above) may be smaller than the other ssDNA molecules of the plurality. For instance, in the complete overlapping strategy, the ssDNA molecules that provide the 5′ and 3′ ends of the second strand of the dsDNA molecule are smaller than the other ssDNA molecules of the plurality.

Each ssDNA molecule in a plurality may be from about 150 to 10000 nucleotides in length, such as about 200 to about 9000 nucleotides in length, about 250 to about 8000 nucleotides in length, about 350 to about 7000 nucleotides in length, about 400 to about 6000 nucleotides in length, about 450 to about 5000 nucleotides in length, about 500 to about 4000 nucleotides in length, about 600 to about 3500 nucleotides in length, about 700 to about 3000 nucleotides in length, about 800 to about 2500 nucleotides in length, about 900 to about 2000 nucleotides in length, about 1000 to about 1500 nucleotides in length, and so on.

Alternatively viewed, each ssDNA molecule in a plurality may comprise at least about 200, 300, 400, 500 nucleotides, such as at least about 600, 700, 800, 900, 1000 nucleotides, e.g. 1100, 1200, 1300, 1400, 1500, 1600, 1700, 1800, 1900, 2000 or more nucleotides.

As noted above, consecutive ssDNA molecules typically will be longer than non-consecutive ssDNA molecules. Thus, for instance, consecutive ssDNA molecules may comprise at least about 500 nucleotides, such as least about 600, 700, 800, 900, 1000 nucleotides, e.g. e.g. 1100, 1200, 1300, 1400, 1500, 1600, 1700, 1800, 1900, 2000 or more nucleotides. In methods that use a mixture of consecutive and non-consecutive ssDNA, the non-consecutive ssDNA molecules typically will be shorter than the consecutive ssDNA molecules, e.g. at least about 150, 200, 300, 400 or 500 nucleotides.

Moreover, even in methods and uses in which all of the ssDNA molecules are consecutive, the ssDNA molecules that provide the 5′ and 3′ ends of one of the strands of the dsDNA may be shorter than the other ssDNA molecules, e.g. by at least the length of the regions of complementarity, e.g. by at least 50 nucleotides, such as by 75, 100, 125, 150 nucleotides or more.

Thus, in the context of the methods and uses provided above (i.e. the complete overlapping and partial overlapping strategies) the ssDNA molecules of (a) (i) may comprise at least about 500 nucleotides, such as at least about 600, 700, 800, 900 or 1000 nucleotides; and at least two of the ssDNA molecules of (a) (ii) may comprise at least about 500 nucleotides, such at least about 600, 700, 800, 900 or 1000 nucleotides.

In the context of other methods and uses provided above (i.e. the minimal overlapping strategy) the ssDNA molecules of (a) (i) may comprise at least about 500 nucleotides, such as at least about 600, 700, 800, 900 or 1000 nucleotides; and the ssDNA molecules of (a) (ii) may be shorter than the ssDNA molecules of (a) (i), such as at least about 150, 200, 300, 400 or 500 nucleotides.

In addition to providing a plurality of ssDNA molecules of sufficient size to produce a dsDNA complex, and subsequently the dsDNA molecule, it will be understood that each of the ssDNA molecules with a non-terminal 5′ end (i.e. the ssDNA molecules that do not provide the 5′ ends of their respective strands) must be phosphorylated to enable ligation to take place. While it is advantageous that each of the ssDNA molecules with a non-terminal 5′ end is provided with a phosphorylated 5′ end, this is not essential as the 5′ ends may be phosphorylated after the assembly of the dsDNA complex. Thus, the method may comprise a further step of phosphorylating the 5′ end(s) of the ssDNA molecules, e.g. using a kinase enzyme, such as T4 polynucleotide kinase.

Similarly, it will be understood that each of the ssDNA molecules with a non-terminal 3′ end (i.e. the ssDNA molecules that do not provide the 3′ ends of their respective strands directly) must be capable of being extended in a polymerase-mediated extension reaction and/or ligated in ligase-mediated ligation reaction, e.g. comprise an extendable and/or ligatable 3′ end (i.e. comprising a functional hydroxyl group).

It may also be useful in some aspects to provide one or both of the ssDNA molecules that provide the 5′ ends of the dsDNA molecule without phosphorylated 5′ ends, e.g. to minimise intermolecular and intramolecular ligation events between dsDNA complexes. For instance, the 5′ ends of one or both of the ssDNA molecules that provide the 5′ ends of the dsDNA molecule may be modified. In this respect, the dsDNA produced by the method and use provided herein may be a linear dsDNA molecule.

Similarly, it may be useful in some aspects to provide one or both of the ssDNA molecules that provide the 3′ ends of the dsDNA molecule (i.e. directly) without extendable and/or ligatable 3′ ends, e.g. to minimise intermolecular ligation events between dsDNA complexes and/or minimise the production undesirable extension products. For instance, the 3′ ends of one or both of the ssDNA molecules that provide the 3′ ends of the dsDNA molecule may be modified. As discussed further below, the dsDNA molecule may be subjected to further steps, e.g. post-synthesis modifications, such as phosphorylation of the 5′ ends to enhance the functionality of the dsDNA molecule, e.g. to facilitate its ligation to other DNA molecules, such as a vector, e.g. plasmid.

It will be understood that in the context of a single stranded DNA molecule, the term “single stranded” refers to a polynucleotide which is single stranded under denaturing conditions, e.g. following the application of heat or suitable chemical denaturing agents, i.e. a polynucleotide with only one continuous backbone (one strand). As noted above, this does not preclude a single stranded DNA molecule from forming secondary or tertiary structures. For example, the single stranded DNA molecule may comprise regions of self-complementarity, and thus may be capable of forming a hairpin or stem-loop structure, mediated by one region of the single stranded DNA molecule hybridising to a complementary region elsewhere in the same single stranded DNA molecule. The terms “single stranded DNA (ssDNA) molecule”, “ssDNA” and “polynucleotide” are used interchangeably herein.

As used herein, the term “plurality” means three or more, e.g. at least 3, 4, 5, 6, 7, 8, 9, 10, 12, 15, 20, 25, 30 or more, such as 40, 50, 75, 100 or more depending on the context of the invention. For instance, a plurality of ssDNA molecules each comprising (e.g. consisting of) a non-overlapping portion of a strand of the dsDNA molecule may contain at least 3 ssDNA molecules, such as 4, 5, 6, 7, 8, 9, 10, 12, 20, 25, 30 or more ssDNA molecules, e.g. 3-50, 4-45, 5-40, 6-35, 7-30, 8-25, 9-20 or 10-15 ssDNA molecules. It will be understood that in some strategies (i.e. the complete overlapping and some of the partial overlapping strategies), one of the strands of the dsDNA is provided by more ssDNA molecules than the other strand, i.e. at least one more ssDNA molecule. In a representative example, where a first strand is provided by four ssDNA molecules, the second strand is provided by five ssDNA molecules. Thus, the number of ssDNA molecules provided in each plurality of ssDNA molecules may be different. However, in some strategies (i.e. the minimal overlapping strategy or some of the partial overlapping strategies) the number of ssDNA molecules provided in each plurality of ssDNA molecules is the same.

It can be seen that if multiple copies (i.e. a plurality) of each plurality of ssDNA molecules is provided in the method and use described herein, this will result in the production of a plurality of dsDNA complexes and, subsequently, dsDNA molecules, e.g. 103, 104, 105, 106, 107, 108, 109, 1010 or more copies of each dsDNA molecule. Thus, the method and use provided herein may be viewed as producing a plurality of dsDNA molecules. In this respect, each copy of a ssDNA molecule that provides a non-overlapping portion of a strand of the dsDNA molecule may be the same, i.e. comprise the same sequence. If the ssDNA molecule contains modified nucleotides, the modified nucleotides in each copy may be the same, i.e. same number, type and location of functional groups in each copy of the ssDNA molecule.

Similarly, where different versions (e.g. sequence variants) of a ssDNA molecule are provided, the term plurality may refer to at least 3 different versions (e.g. 3 sequence variants), such as 4, 5, 6, 7, 8, 9, 10, 12, 20, 25, 30 or more different versions, wherein a plurality of copies of each version may be provided (e.g. 103, 104, 105, 106, 107, 108, 109, 1010 or more copies of each version). Where different versions are used to make a library of dsDNA molecules a gene library, such as for phage display) the term plurality may refer to at least 100 (e.g. 103, 104, 105, 106, 107, 108, 109, 1010 or more versions), wherein a plurality of copies of each version may be provided (e.g. 103, 104, 105, 106, 107, 108, 109, 1010 or more copies of each version).

As noted above, it is hypothesised that the use of very pure and correct ssDNA molecules contributes to the efficacy of the method and use provided herein. In this respect, it is well-known in the art that polynucleotides produced using solid phase chemical synthesis methods (e.g. phosphoramidite synthesis) commonly contain impurities (e.g. by-products of the chemical synthesis reactions) and incorrect sequences, particularly for polynucleotides containing more than 50 nucleotides. In contrast, polynucleotides produced using enzymatic synthesis methods, particularly using so-called high fidelity DNA polymerases, readily can be highly purified and contain minimal sequence errors.

Thus, the ssDNA molecules provided in the method and use described herein are not produced using chemical synthesis methods, e.g. solid phase chemical synthesis methods, such as phosphoramidite synthesis.

Alternatively viewed, each ssDNA molecule provided in the method and use described herein is produced via enzymatic synthesis, i.e. enzymatically-produced ssDNA molecules. Numerous methods for the enzymatic production of ssDNA molecules are known in the art, e.g. asymmetric PCR (e.g. LATE-PCR), Monoclonal Stoichiometric (MOSIC) polynucleotide production (Ducani et al. 2013, Nature Methods, 647-652, incorporated herein by reference), terminal deoxynucleotidyl transferase (TdT) synthesis etc. It will be understood that any suitable enzymatic synthesis method may be used to produce the ssDNA molecules for use in the method and use provided herein. Thus, the ssDNA molecules for use in the method and use provided herein may be produced using asymmetric PCR.

While it is known that PCR based synthesis methods may result in polynucleotides that contain errors, the likelihood of an error occurring in a polynucleotide containing about 1000 nucleotides or less is low. Thus, when a ssDNA molecule is produced using asymmetric PCR, it may contain 1000 nucleotides or less. However, it will be understood that longer ssDNA molecules (e.g. containing more than 1000 nucleotides) may be synthesised accurately using a high-fidelity polymerase, e.g. Thermococcus litoralis DNA polymerase (Vent) as described in Perler et al., Proc. Natl. Acad. Sci. USA (1992) 89:5577-5581; Pyrococcus species GB-D (Deep Vent); Pyrococcus furiosus DNA polymerase (Pfu) as described in Lundberg et al., Gene (1991) 108:1-6, Pyrococcus woesei (Pwo) and the like.

Thus, the enzymatic synthesis (e.g. asymmetric PCR) method may use a high-fidelity polymerase, i.e. a polymerase with an error rate of 2.0×10⁻⁵ or less, such as 1.0×10⁻⁵ or less, 9.0×10⁻⁶ or less, 8.0×10⁻⁶ or less, or 7.0×10⁻⁶ or less.

The “error rate” (f) is calculated as f=n/S (target size×d), where n is the number of mutations observed for all polynucleotides that were sequenced and the (target size×d) for each polynucleotide that was cloned, and wherein (d) is the average of doubling values for each PCR reaction, where doublings are calculated from the formula 2d=(ng DNA after PCR/ng DNA input).

While any method of enzymatic synthesis may be used to produce ssDNA molecules for use in the method provided herein, the ssDNA molecules produced using methods that do not involve PCR may be preferred to avoid the introduction of sequence errors, especially for the production of long ssDNA molecules (e.g. comprising at least about 500 nucleotides, e.g. 1000 nucleotides or more). For instance, the ssDNA molecules may be produced using Rolling Circle Amplification (RCA)-based methods, particularly the MOSIC method. Methods for producing ssDNA molecules using the MOSIC method are described in Ducani et al. (supra), WO2020/161187 and WO2021/170656 (all hereby incorporated by reference).

Thus, a ssDNA molecule for use in the method described herein may be produced by a method comprising:
(a) providing a circular DNA molecule comprising a nucleotide sequence encoding the ssDNA molecule bordered by cleavage domains;
(b) performing a rolling circle amplification (RCA) reaction with the circular DNA molecule of (a) as a template; and
(c) cleaving (e.g. enzymatically cleaving) the product of the RCA reaction at the cleavage domains to release a plurality of copies of the ssDNA molecule; and optionally
(d) isolating or purifying the ssDNA molecules produced in (c).

The step of providing a circular DNA molecule may be achieved by any suitable means and may depend on the structure of the circular DNA molecule. For instance, the circular DNA molecule may be provided via a minicircle plasmid as described in WO2021/170656 (hereby incorporated by reference). In this respect, the circular DNA molecule may be a single stranded DNA molecule or a double stranded DNA molecule.

In embodiments where the circular DNA molecule is a double stranded molecule, it will be evident that the circular DNA molecule must be processed to provide an RCA template. Thus, in some embodiments, the method comprises an additional step of cleaving a single strand of the circular DNA molecule to provide an RCA template, before the RCA reaction is performed.

Thus, step (a) of the method provided herein may include the method described above for producing a ssDNA molecule of the plurality of ssDNA molecules. Step (a) of the method provided herein may include the method described above for producing all of the plurality of ssDNA molecules used in the method provided herein. It will be understood that each of the plurality of ssDNA molecules may be produced in separate reactions and subsequently pooled or combined to form the plurality of ssDNA molecules for one or both strands. Alternatively, the plurality of ssDNA molecules for one or both strands may be produced in a single reaction mixture in a so-called multiplex reaction. For instance, as shown in the Examples, each ssDNA molecule may be encoded by a separate circular DNA molecule which can be combined in a single reaction mixture such that the ssDNA molecules are produced simultaneously.

It will be understood that the circular DNA molecule may comprise a plurality of nucleotide sequences each encoding a ssDNA molecule provided in (a) bordered by cleavage domains, such that more than one ssDNA molecule of the plurality of ssDNA molecules, e.g. all of the plurality of ssDNA molecules of a strand of the dsDNA molecule, may be provided in a single step.

The ssDNA molecules for one or both strands may be produced by a combination of the multiplex reaction strategy described above and the circular DNA molecule comprising a plurality of nucleotide sequences each encoding a ssDNA molecule.

It will be appreciated that each ssDNA molecule used in the method and use provided herein may be provided separately and therefore may be produced using different methods. Thus, for instance, some ssDNA molecules may be produced by asymmetric PCR and/or TdT synthesis and some via the MOSIC method, and subsequently combined (i.e. mixed) to provide the plurality of ssDNA molecules. However, to avoid additional steps, it may be preferable to produce all of the ssDNA molecules by the same method, e.g. by the MOSIC method described above.

As shown in the Examples, it has been unexpectedly demonstrated that all of the ssDNA molecules for both strands of the dsDNA molecule may be produced and assembled in a single reaction mixture, i.e. a so-called “one-pot” reaction. This is a particularly advantageous as it reduces the complexity of the method and the consumables used in the process, thereby reducing time and costs. Thus, all of the steps of the methods, including steps of producing the ssDNA molecules, may be performed in a single reaction mixture and/or vessel. In other words, the method provided herein may be a one-pot reaction.

The term “cleavage domain” as used herein typically refers to a domain within the circular DNA molecule that results in a domain within the RCA product that can be cleaved specifically to release the ssDNA molecules. Thus, the cleavage domain in the circular DNA molecule may be capable of cleavage directly or it may simply encode a cleavage domain that is only functional in the RCA product or functional under specific conditions, e.g. upon contact with a co-factor.

In some embodiments, a cleavage domain comprises or consists of a sequence capable of forming a hairpin structure. A hairpin structure may also be known as a hairpin-loop or a stem-loop and these terms are used interchangeably herein. A hairpin is an intramolecular base-pairing pattern that can occur in a single-stranded DNA or RNA molecule. A hairpin occurs when two regions of the same strand, usually complementary in nucleotide sequence when read in opposite directions, base-pair (hybridise) to form a double stranded stem (a duplex) and an unpaired, i.e. single-stranded, loop. The resulting structure can be described as lollipop-shaped.

Thus, in some embodiments where the cleavage domain comprises or consists of a sequence capable of forming a hairpin structure, the cleavage domain comprises sequences that are self-complementary. As the RCA product extends, the hybridization of these self-complementary regions results in a hairpin structures, wherein the double-stranded portion of the hairpin structure comprises a sequence that is recognised by a cleavage enzyme. Thus, cleavage of the double-stranded portion of the hairpin structures in the RCA product results in the release of the ssDNA molecules and hairpin structures (i.e. oligonucleotides that form the hairpin structures).

“Cleavage” includes any means of breaking a covalent bond. Thus, in the context of the invention, cleavage involves cleavage of a covalent bond in a nucleotide chain (i.e. strand cleavage or strand scission), for example by cleavage of a phosphodiester bond.

Thus, in some embodiments, a cleavage domain may comprise a sequence that is recognised by one or more enzymes capable of cleaving a nucleic acid molecule, i.e. capable of breaking the phosphodiester linkage between two or more nucleotides. For instance, a cleavage domain may comprise a restriction endonuclease (restriction enzyme) recognition sequence. Restriction enzymes cut double-stranded or single stranded DNA at specific recognition nucleotide sequences known as restriction sites, and suitable enzymes are well-known in the art. For example, it may be particularly advantageous to use rare-cutting restriction enzymes, i.e. enzymes with a long recognition site (at least 8 base pairs in length), to facilitate the design of the polynucleotide sequence(s) in the circular DNA molecule, e.g. to avoid the inclusion of a cleavage recognition site that occurs within the sequence of the ssDNA molecule.

A cleavage domain may comprise a sequence that is recognised by a type II restriction endonuclease, more preferably a type IIs restriction endonuclease. While any suitable cleavage domain and cleavage enzyme may be used, the cleavage enzyme that recognises the cleavage domains bordering the polynucleotide sequences may be BseGI, BtsCI or an isoschizomer thereof, e.g. BstF5I or Fokl. Other representative enzymes that may be used include BsrDI, Btsl, BtsIMutl, Mlyl or isoschizomers thereof.

As discussed above, the method provided herein may utilise long ssDNA molecules, e.g. at least about 500, 750 or 1000 nucleotides in length. It will be understood that longer sequences are more likely to contain sequences recognised by endonucleases, particularly type IIs restriction endonucleases. Thus, the cleavage domains that border the polynucleotide may be homing endonuclease cleavage domains, i.e. the cleavage enzyme used in the cleavage step may be homing endonucleases. For instance, the cleavage domains that border the polynucleotide may be meganuclease cleavage domains, i.e. the cleavage enzyme used in the cleavage step may be a meganuclease.

The term “meganuclease” refers to endonucleases characterized by a large recognition site, e.g. double-stranded DNA sequences of 12 to 40 base pairs. Thus, many homing endonucleases, such as I-Scel, may be viewed as meganucleases. Chimeric meganucleases may be produced by fusing a nucleic acid binding domain and an endonuclease cleavage domain from different proteins. For instance, any protein domain capable of site-specific recognition (binding) of a DNA sequence as described above may be fused to a cleavage domain from an endonuclease that cleaves outside of the sequence recognised by the endonuclease, e.g. at a specific distance from the recognition sequence. Any suitable meganuclease known in the art may be used in the methods described herein, e.g. in the step of cleaving the RCA product.

Thus, the step of cleaving the product of the RCA reaction may comprises contacting the RCA product with a cleavage enzyme under suitable conditions to selectively cleave the RCA product in the cleavage domains.

Upon cleavage of the RCA product, the ssDNA polynucleotides are released. The polynucleotides that are released may consist only of the polynucleotide sequence (i.e. with no additional nucleotides), or they may comprise one or more additional nucleotides from the cleavage domains that border the polynucleotide sequences at one or both ends. Thus, the ssDNA polynucleotides may comprise 1, 2, 3, 4, 5, 6, 7, 8, 9 or 10 or more nucleotides from the cleavage domains at one or both ends. Preferably, the sequence of the circular DNA molecule is designed such that when the RCA product is cleaved, the polynucleotides that are released do not contain any additional nucleotides from the cleavage domains. Alternatively viewed, the cleavage domains that border the polynucleotide sequence(s) may be arranged in the circular DNA molecule such that their cleavage in the RCA product results in the release of ssDNA polynucleotides that do not contain any additional nucleotides from the cleavage domains.

As cleavage enzymes may cleave a nucleic acid molecule at a position outside of the cleavage enzyme recognition sequence, there may be one or more nucleotides between a cleavage domain and a polynucleotide sequence. Alternatively viewed, the cleavage domain may contain nucleotide sequences in addition to the cleavage enzyme recognition sequence to ensure that cleavage results in the release of the complete ssDNA polynucleotides, preferably without any additional nucleotides (e.g. nucleotides that form part of the cleavage domains).

Thus, the term “bordered”, in the context of a polynucleotide sequence bordered by cleavage domains, refers to cleavage domains that are directly or indirectly adjacent to the polynucleotide sequence. Alternatively viewed, the cleavage domains are positioned at either end of the polynucleotide sequence, i.e. the cleavage domains are upstream and downstream (at the 5′ and 3′ ends) of the polynucleotide sequence. The cleavage site of the cleavage domains (e.g. the site at which a cleavage enzyme cleaves a cleavage domain) may be directly adjacent to the end of the polynucleotide sequence it borders. However, the polynucleotide sequence and cleavage domain sequence may overlap, i.e. the end of the polynucleotide sequence may form part of the cleavage domain, e.g. when the cleavage site is an internal site within the cleavage domain, i.e. the cleavage domains may form the ends or part of the ends of the polynucleotide sequence. Thus, there may be one or more, e.g. 1, 2, 3, 4, 5, 6, 7, 8, 9 or 10 or more, nucleotides between the cleavage domain and the polynucleotide sequence (i.e. between the ends of the sequences). However, the cleavage domain and the polynucleotide sequence may overlap one or more, e.g. 1, 2, 3, 4, 5, 6, 7, 8, 9 or 10 or more, nucleotides.

The size of the cleavage domains in the circular DNA molecule is not particularly limited and will depend on the type of cleavage domain, as described above. The relative lengths of the polynucleotide sequences and the cleavage domains are designed or selected such that they are different from each other, to allow for the ssDNA polynucleotides to be easily purified once the RCA product has been cleaved at the cleavage domain. The cleavage domain(s) may thus be selected to be shorter than the polynucleotide sequence(s) in the circular DNA molecule. If the circular DNA molecule contains a plurality of polynucleotide sequences of different lengths, the cleavage domain(s) may be selected to be shorter than the shortest polynucleotide sequence in the circular DNA molecule.

Cleavage domains may be about 4-50 nucleotides in length, such as about 5-45, 6-40, 7-35 or 8-30 nucleotides in length. In some embodiments they may range from about 10 to 25 nucleotides in length, including from about 12 to 22 or from about 14 to 20. However, it will be evident that any suitable length of cleavage domain may be used in the invention as long as it meets the functional requirements described above. Thus, longer cleavage domains are contemplated, e.g. from about 22 to 70 nucleotides in length, including from about 25 to 60 or from about 30 to 55 nucleotides in length. Alternatively, cleavage domains may range from about 35 to 80 nucleotides in length, including from about 40 to 70 or from about 45 to 60 nucleotides in length.

The cleavage domains that border the polynucleotide sequences may be the same or different from each other. Advantageously, the cleavage domains that border the polynucleotide sequences are the same such that a single cleavage step is sufficient to release all of the single stranded DNA polynucleotides. However, as mentioned above, some cleavage enzymes may cleave a nucleic acid molecule at a position outside of the cleavage enzyme recognition sequence or may recognise more than one sequence (e.g. where variation within the enzyme recognition sequence is allowed). Thus, it is not necessary for the whole sequence of the cleavage domains to be the same in order for them to be cleaved by the same enzyme. For instance, the step of cleaving the RCA product may comprise contacting the RCA product with a single cleavage enzyme under conditions suitable to cleave the cleavage domains in the RCA product.

Suitable conditions to cleave the cleavage domains in the RCA product will be dependent on the means used to achieve cleavage. For instance, where cleavage is achieved using a cleavage enzyme, such as a restriction endonuclease or homing endonuclease, conditions will differ depending on the enzyme selected, and suitable conditions are well-known in the art, e.g. the cleavage step may follow the manufacturer's instructions.

For instance, a cleavage enzyme, e.g. a restriction or homing endonuclease, may specifically bind to its cleavage recognition site and selectively (e.g. specifically) cleave the nucleic acid in a variety of buffers, such as phosphate buffered saline (PBS), 4-(2-hydroxyethyl)-1-piperazineethanesulfonic acid (HEPES), HEPES buffered saline (HBS), and Tris buffered saline (TBS), both with and without EDTA. Cleavage may occur in a pH range of about 3.0-10.0, e.g. 4.0-9.0, 5.0-8.0, over a wide range of temperatures, e.g. 0-70° C. The skilled person would readily be able to determine other suitable conditions.

Thus, the method for producing a ssDNA molecule may comprise a step of performing rolling circle amplification using a circular DNA molecule as a template. Rolling-circle amplification (RCA) is well known in the art, being described in Dean et al., 2001 (Rapid Amplification of Plasmid and Phage DNA Using Phi29 DNA Polymerase and Multiply-Primed Rolling Circle Amplification, Genome Research, 11, pp. 1095-1099), the disclosures of which are herein incorporated by reference. In brief, RCA relates to the synthesis of nucleic acid molecules using a circular single stranded nucleic acid molecule, e.g. a circle or circular oligonucleotide, as rolling circle template (a RCA template) and a strand-displacing polymerase to extend a primer which is hybridised to the template. The addition of a polymerase and nucleotides starts the synthesis reaction, i.e. polymerization. As the rolling circle template is endless, the resultant product is a long single stranded nucleic acid molecule composed of tandem repeats that are complementary to the rolling circle template.

A typical RCA reaction mixture includes a circular DNA molecule acting as a template, and one or more primers that are employed in the primer extension reaction, e.g. RCA may be templated by a single primer to generate a single concatemeric product or multiple primers, each annealing to a different region of the circular template to produce multiple concatemeric products per circle. The oligonucleotide primers with which the circular DNA molecule may be contacted will be of sufficient length to provide for hybridization to the circular DNA molecule under annealing conditions. It will be understood that the primer may be provided by cleaving (e.g. nicking) a single strand of a double stranded circular DNA molecule provided in step (a).

The RCA reaction mixture may further include an aqueous buffer medium that includes a source of monovalent ions, a source of divalent cations and a buffering agent. Any convenient source of monovalent ions, such as KCl, K-acetate, NH₄-acetate, K-glutamate, NH₄Cl, ammonium sulphate, and the like may be employed. The divalent cation may be magnesium, manganese, zinc and the like, where the cation will typically be magnesium. Any convenient source of magnesium cation may be employed, including MgCl₂, Mg-acetate, and the like. The amount of Mg²⁺ present in the buffer may range from 0.5 to 10 mM, but will preferably range from about 3 to 6 mM, and will ideally be at about 5 mM. Representative buffering agents or salts that may be present in the buffer include Tris, Tricine, HEPES, MOPS and the like, where the amount of buffering agent will typically range from about 5 to 150 mM, usually from about 10 to 100 mM, and more usually from about 20 to 50 mM, where the buffering agent may be present in an amount sufficient to provide a pH ranging from about 6.0 to 9.5. Other agents which may be present in the buffer medium include chelating agents, such as EDTA, EGTA and the like.

In addition to the above components, the reaction mixture typically includes a polymerase (defined further below, e.g. phi29 DNA polymerase), one or more nucleotides and other components required for a DNA polymerase reaction as described below. The desired polymerase activity may be provided by one or more distinct polymerase enzymes.

The RCA reaction ultimately produces a polynucleotide product comprising adjacent (tandem) repeats of the complementary sequence of the circular DNA molecule. This product may be known as a concatemer, an RCA product or “RCP”. The RCA product therefore comprises a linear sequence made up of polynucleotide sequences (or more particularly the reverse complement of the polynucleotide sequences of the circular DNA molecule template) bordered by cleavage domains.

Any DNA polymerase with at least some strand displacement activity may be used in the RCA reaction used to produce the ssDNA molecules for use in the method provided herein. Strand displacement activity ensures that once the polymerase has extended around the circular DNA molecule, it can displace the primer sequence and the elongating product and continue to “roll” around the template. In embodiments where the nicked strand of the DNA minicircle provides the primer for RCA extension, the strand displacement activity ensures that the polymerase can displace the nicked strand. Suitable DNA polymerase enzymes with at least some strand displacement activity include phi29 DNA polymerase, E. coli DNA polymerase I, Bsu DNA polymerase (large fragment), Bst DNA polymerase (large fragment) and Klenow fragment. As used herein, the term “DNA polymerase” includes not only naturally occurring enzymes but also all such modified derivatives, including also derivatives of naturally occurring DNA polymerase enzymes. For instance, in some embodiments, the DNA polymerase may have been modified to remove 5′-3′ exonuclease activity.

Particularly preferred DNA polymerase enzymes for use in the RCA reaction include phi29 DNA polymerase, Bst DNA polymerase and derivatives, e.g. sequence-modified derivatives, or mutants thereof.

Sequence-modified derivatives or mutants of DNA polymerase enzymes include mutants that retain at least some of the functional activity, e.g. DNA polymerase activity and at least some strand displacement activity, of the wild-type sequence. Mutations may affect the activity profile of the enzymes, e.g. enhance or reduce the rate of polymerisation, under different reaction conditions, e.g. temperature, template concentration, primer concentration etc. Mutations or sequence-modifications may also affect the exonuclease activity and/or thermostability of the enzyme.

As noted above, the RCA reaction may be conducted using conventional nucleotides, functionalised nucleotides, or a mixture of conventional and functionalised nucleotides.

The term “release” is used in the present context to refer to cleaving the RCA product at the cleavage domains bordering the polynucleotide sequences so as to detach or separate the polynucleotides from the cleavage domains. It is desirable that the release of a given polynucleotide will involve cleavage at both cleavage domains bordering the polynucleotide sequence.

It is not necessary for cleavage to occur at all of the cleavage domains in the RCA product in order to generate the single stranded DNA polynucleotides. The cleavage of a portion of cleavage domains will still result in the release of a portion of single stranded DNA polynucleotides. Thus, the step of cleaving the RCA product may result in cleavage of at least about 30% of the cleavage domains in the RCA product, e.g. at least about 35%, 40%, 45%, 50%, 60%, 70% or 80%. However, the step of cleaving the RCA product may result in cleavage of at least about 90% of the cleavage domains in the RCA product, e.g. 95% or more.

Alternatively viewed, the step of cleaving the RCA product may result in the release of at least about 30% of the single stranded DNA polynucleotides contained in the RCA product, e.g. at least about 35%, 40%, 45%, 50%, 60%, 70% or 80%. Moreover, the step of cleaving the RCA product may result in the release of at least about 90% of the single stranded DNA polynucleotides contained in the RCA product, e.g. 95% or more.

Once the single stranded DNA polynucleotides have been released, it may be desirable to isolate, separate or purify the single stranded DNA polynucleotides from the cleavage reaction mixture (e.g. reaction components and/or degradation products such as cleavage domains, uncleaved RCA products etc.) to provide the ssDNA molecules for use in the method and use provided herein.

Thus, the method of producing ssDNA molecules may further comprise a step of isolating, separating or purifying the single stranded DNA polynucleotides. This isolation, separation or purification may be done by any suitable method known in the art.

Following the isolation, separation or purification step the single stranded DNA polynucleotides are preferably substantially free of any contaminating components derived from the materials or components used in the isolation procedure or in their preparation (e.g. reaction components and/or degradation products such as cleavage domains, uncleaved RCA products etc.). The single stranded DNA polynucleotides are purified to a degree of purity of more than about 70, 80 or 90%, such as more than about 95 or 99% purity as assessed w/w (dry weight). Such purity levels may include degradation products of the single stranded DNA polynucleotides.

For example, the products of the cleavage reaction may be separated by size using gel electrophoresis using an agarose gel or a polyacrylamide gel. The desired polynucleotides can then be isolated from the gel and purified further, if necessary, according to methods known in the art. Other methods for purifying, isolating or separating the polynucleotides of the invention utilise chromatography (e.g. HPLC, size-exclusion, ion-exchange, affinity, hydrophobic interaction, reverse-phase) or capillary electrophoresis.

However, it will be understood that in some embodiments, e.g. the “one-pot” embodiments, isolation, separation or purification of the ssDNA molecules is not required or desirable. Thus, in some embodiments, the ssDNA molecules are not isolated, separated or purified prior to the step of hybridizing the ssDNA molecules.

The method and use provided herein utilise regions of complementarity between the ssDNA molecules in each plurality of ssDNA molecules to form a DNA complex. Each ssDNA molecule of a plurality of ssDNA molecules (i.e. a plurality of ssDNA molecules that provides all the portions of a strand of the dsDNA molecule) comprises a region of complementarity to at least one ssDNA molecule of the other plurality of ssDNA molecules. The regions of complementarity typically include the “end regions” of the ssDNA molecules, i.e. the regions of the ssDNA molecules that contain the 5′ or 3′ end.

It will be understood that the terminal ssDNA molecules of one strand (i.e. the ssDNA molecules that comprise the 5′ or 3′ of a strand of the dsDNA molecule) will have a sequence that is complementary to only one ssDNA molecule of the other strand. However, the internal ssDNA molecules (i.e. the ssDNA molecules that do not comprise the 5′ or 3′ of a strand of the dsDNA molecule), and the terminal ssDNA molecules of the other strand, will have two regions of complementarity, i.e. a first region that is fully complementary to an end region of a ssDNA molecule of the opposite strand and a second region that is fully complementary to an end region of a different ssDNA molecule of the opposite strand.

The two regions of complementarity of a ssDNA molecule (e.g. an internal ssDNA molecule) may be directly adjacent to each other, such that there is no intervening nucleotide sequence between the regions (e.g. as in the complete overlapping strategy). This means that 5′ and 3′ ends of the ssDNA molecules of the opposite strand that hybridise to the ssDNA molecule are directly adjacent to each other, i.e. there is no gap between the 5′ and 3′ ends.

However, in other strategies, the two regions of complementarity of a ssDNA molecule (e.g. an internal ssDNA molecule) may be indirectly adjacent to each other, such that there is an intervening nucleotide sequence between the regions (e.g. as in the partial overlapping strategy). This means that 5′ and 3′ ends of the ssDNA molecules of the opposite strand that hybridise to the ssDNA molecule are indirectly adjacent to each other, such that there is a gap between the 5′ and 3′ ends which must be filled-in before the ssDNA molecules can be ligated.

The ssDNA molecules of each plurality of ssDNA molecules are designed such that the regions of complementarity hybridise to ssDNA molecules of the opposite strand that are adjacent portions of that strand, such that when the ssDNA molecules are ligated the portions of each strand are joined in the correct order to produce the dsDNA molecule. As noted above, the ssDNA molecules may hybridise to the ssDNA molecule of the opposite strand such that they are directly adjacent (without a gap) or indirectly adjacent (i.e. with a gap). ssDNA molecules that are directly adjacent may be considered to form a junction between the two molecules (e.g. a nick site), such that the molecules may be joined in a ligase-mediated ligation reaction.

The term “region” refers to a part of a ssDNA molecule.

As discussed above, it is preferred that the regions of the ssDNA molecules that hybridise to the ssDNA molecules of the opposite strand are fully complementary as defined above.

Whilst not wishing to be bound by theory, it is hypothesised that the size of the regions of complementarity contributes to the efficacy of the method and use provided herein. Thus, the regions of the ssDNA molecules that are complementary to the ssDNA molecules of the opposite strand must contain at least 50 nucleotides, such as at least about 60, 70, 80, 90 or 100 nucleotides. The regions of complementary may comprise at least about 110, 125, 150, 200, 250, 300, 400 or 500 nucleotides. Thus, for instance, the regions of complementary may comprise 50-750, 60-700, 70-650, 80-600, 90-550 or 100-500 nucleotides, such as 150-500, 200-500 or 250-500 nucleotides.

The ssDNA molecules are provided in equimolar (i.e. stoichiometric 1:1:1 etc.) amounts, such that approximately the same number of each ssDNA molecule is present. It is thought that this may reduce the formation of undesired dsDNA complexes. Thus, when different versions of a ssDNA molecule are provided (e.g. sequence variants of the ssDNA molecule) the total concentration of the different versions is equimolar to the concentration of the other ssDNA molecules in the plurality, i.e. each version of a ssDNA molecule is provided at a lower concentration than ssDNA molecules for which only one version is provided.

The ssDNA molecules may be provided by any suitable method or means. For instance, each plurality of ssDNA molecules may be combined separately to provide two separate mixtures of ssDNA molecules (i.e. one for each strand), which are subsequently combined, i.e. to enable hybridisation to occur. Alternatively, all of the ssDNA molecules may be combined in a single mixture in any order and under any appropriate conditions, e.g. conditions suitable to maintain the integrity of the ssDNA molecules, i.e. to avoid degradation of the ssDNA molecules, e.g. in a buffered solution, e.g. 20-100 mM Tris-HCl, pH 7-9, optionally comprising a chelating agent, such as EDTA or EGTA.

Thus, the method and use provided herein may involve a step of combining or mixing the ssDNA molecules, i.e. to provide a mixture of ssDNA molecules. However, as noted above, the ssDNA molecules may be produced in a single reaction mixture and in these embodiments, a step of combining or mixing the ssDNA molecules is not required.

The method and use involve hybridizing the ssDNA molecules that contain regions of complementarity to produce a dsDNA complex comprising all of the ssDNA molecules. This step may be viewed as a step of assembling the dsDNA complex. The step of hybridising involves subjecting the mixture of ssDNA molecules to annealing conditions. It will be evident that the method and use provided herein involves a single hybridization step and followed by a single ligation step, i.e. all of the ssDNA molecules that form the dsDNA molecule are combined or mixed (present in the mixture) prior to hybridisation and ligation. Thus, the method and use does not involve sequential hybridisation and/or sequential ligation steps or multistep hybridisation and/or ligation steps, e.g, wherein a first subset of ssDNA molecules is hybridised and ligated to form a partial dsDNA molecule that is used in subsequent hybridisation and ligation steps to form the full dsDNA molecule.

The term “annealing conditions” refers to the conditions under which two nucleic acid molecules comprising complementary nucleotide sequences will specifically hybridise to each other. Various parameters affect hybridisation including temperature, salt concentration, nucleic acid concentration, composition and length, and buffer composition. The skilled person readily can determine suitable annealing conditions for a particular combination of ssDNA molecules as a matter of routine.

In a representative example, the step of hybridizing the ssDNA molecules to produce a dsDNA complex comprises subjecting the mixture of ssDNA molecules (i.e. all of the ssDNA molecules) to a temperature suitable to denature DNA molecules, e.g. heating the mixture to at least 94° C., such as 94-100° C. (e.g. 94, 95, 96, 97, 98 or 99° C.), and subsequently cooling the mixture to a temperature that enables the ssDNA molecules to specifically hybridise to each other, e.g. cooling the mixture to less than 40° C., such as 4-40° C. (e.g. 10-35, 15-30, 20-25° C., such as room temperature). As discussed above, the method and use provided herein advantageously uses long regions of complementarity. Accordingly, it will be understood that the ssDNA molecules may hybridise to each other at relatively high temperatures, e.g. as high as about 85° C. Thus, cooling the mixture to a temperature that enable the ssDNA molecules to specifically hybridise to each other may involve cooling the mixture to less than about 90° C., such as about 40-90° C. (e.g. about 50-89° C., 60-88° C., 65-87° C., 70-86° C. or 75-85° C.). A further cooling step may be required for the subsequent extension and/or ligation step, i.e. to a temperature at which the polymerase and/or ligation enzyme is active (e.g. to avoid denaturing the enzyme(s)). However, further cooling may not be needed when the extension and/or ligation step uses a thermostable polymerases and/or ligase, such as Taq polymerase and/or Taq ligase (or functional variants thereof).

Thus, the method and use provided herein may involve steps of heating and cooling ssDNA molecules (i.e. the mixture of ssDNA molecules) to enable hybridization of the ssDNA molecules that contain regions of complementarity, thereby producing a dsDNA complex.

The term “hybridisation” or “hybridises” as used herein refers to the formation of a duplex between nucleotide sequences which are sufficiently complementary to form duplexes via Watson-Crick base pairing.

The term “dsDNA complex” refers to a dsDNA molecule in which each strand is composed of more than one ssDNA. Alternatively viewed, it refers to plurality of ssDNA molecules that are bound non-covalently (i.e. hybridised) to form a dsDNA molecule in which the phosphodiester backbone of one or both strands is not completely joined, i.e. one or both strands contains more than one 5′ and 3′ end available for reaction.

A dsDNA complex may be viewed as a “fully double stranded” or “completely double stranded” DNA complex when all of the ssDNA molecules in each strand are directly adjacent to each other, i.e. the ssDNA molecules of each strand form ligatable junctions. Thus, a fully double stranded DNA complex may be viewed as a “nicked dsDNA”, i.e. containing one or more breaks in the phosphodiester backbone.

A dsDNA complex may be viewed as a “partially double stranded” DNA complex when two or more of the ssDNA molecules in a strand are indirectly adjacent to each other, i.e. the ssDNA molecules of at least one strand form gaps in the strand.

Thus, the step of hybridising the ssDNA molecules may result in a fully dsDNA complex (e.g. the completely overlapping strategy) or a partially dsDNA complex (e.g. the partially or minimal overlapping strategy). The step of hybridising the ssDNA molecules may be viewed as assembling the dsDNA complex.

It will be evident that when the ssDNA molecules are non-covalently bound by their regions of complementarity (i.e. hybridised) in the dsDNA complex, they are no longer fully single stranded (they may be partially single stranded). However, in the context of the method and use provided herein, it is convenient to refer to them as ssDNA molecules on the basis that they would form discrete ssDNA portions of the dsDNA molecule under denaturing conditions.

When the step of hybridising the ssDNA molecules results in the formation (i.e. assembly) of a partially dsDNA complex, the gaps between the ssDNA molecules must be “filled-in” (to produce a fully dsDNA complex) before the ssDNA molecules can be ligated, i.e. joined in a ligase-mediated ligation reaction.

The gaps between the ssDNA molecules in a partially dsDNA complex are “filled in” by a polymerase enzyme using the ssDNA molecules of the opposite stand as a template. Thus, the method provided herein may comprise a step of extending the 3′ ends of the ssDNA molecules to produce a fully dsDNA complex. It will be understood that the extension step involves the extension of the internal 3′ ends of the ssDNA molecules, i.e. the templated extension of the 3′ ends. In other words, the 3′ ends of the ssDNA molecules that form the 3′ end of the dsDNA molecule (i.e. the terminal 3′ ends) are not extended in the extension step. As the nucleotide sequences of ssDNA molecules opposite the gaps may be different, i.e. by virtue of providing a plurality of versions of a ssDNA molecule in the reaction mixture, the polymerase-mediated extension reaction may facilitate the production of a mixture of dsDNA molecules (i.e. comprising different sequences).

Any suitable DNA polymerase may be used in the extension step and readily may be selected by a person of skill in the art as a matter of routine. In this respect, it will be evident that the polymerase enzyme should not have strand displacement activity or 5′ exonuclease activity so as to avoid displacing or degrading ssDNA molecules of the partially dsDNA complex downstream (3′ to) the ssDNA molecules that provide the 5′ ends of dsDNA molecule. For instance, the DNA polymerase may be T4 DNA polymerase, Klenow fragment of DNA polymerase I, Taq polymerase or sequence-modified derivatives, or mutants thereof. The polymerase may be a thermostable polymerase and/or a high-fidelity polymerase, such as the Q5@ High-Fidelity DNA Polymerase (NEB). As such, the polymerase-mediated extension step may be performed at a relatively high temperature, such as between 55-80° C., e.g. 60-75° C. or 65-75° C. This may involve increasing the temperature of the reaction mixture containing the dsDNA complex, i.e. a heating step following the hybridisation step (i.e. following assembly of the dsDNA complex). The extension step may be performed simultaneously with the hybridisation step.

Sequence-modified derivatives or mutants of DNA polymerase enzymes include mutants that retain at least some of the functional activity, e.g. DNA polymerase activity, of the wild-type sequence. Mutations may affect the activity profile of the enzymes, e.g. enhance or reduce the rate of polymerisation, under different reaction conditions, e.g. temperature, template concentration, primer concentration etc. Mutations or sequence-modifications may also affect the exonuclease activity and/or thermostability of the enzyme.

Following the formation of the fully dsDNA complex, i.e. after the hybridising and extending steps (if applicable), the ssDNA molecules are ligated to produce (synthesise, form etc.) the dsDNA molecule. Alternatively viewed, the hybridising and extension steps (if applicable) result in the formation of ligatable junctions, wherein the ssDNA molecules are directly adjacent to each other and held in place by regions of complementarity on ssDNA molecules of the opposite strand. The step of ligating the adjacent ssDNA molecule may be achieved by any suitable enzymatic means, i.e. addition of a ligase enzyme under appropriate conditions. As discussed further below, the ligase activity (e.g. ligase enzyme) may be provided in a host cell containing the dsDNA complex, i.e. when the dsDNA complex is introduced into a host cell (e.g. a bacterial cell) in conjugation with a linearised nucleic acid vector (e.g. plasmid).

Thus, the method and use provided herein does not include chemical ligation, particularly conjugating adjacent nucleotides via click chemistry, a CuAAC reaction between adjacent terminal cytidine and thymidine residues giving rise to a 1,4-linked 1.2,3-triazole. However, as noted elsewhere herein, the dsDNA molecule produced by the method may be subjected to click-chemistry reactions in subsequent steps.

As is known in the art, ligases catalyze the formation of a phosphodiester bond between juxtaposed 3′-hydroxyl and 5′-phosphate termini of two immediately adjacent nucleic acids. Thus, a ligase may be viewed as any enzyme capable of catalysing this reaction. Any convenient ligase may be employed, where representative ligases of interest include, but are not limited to: Temperature sensitive and thermostable ligases. Temperature sensitive ligases include, but are not limited to, bacteriophage T4 DNA ligase, bacteriophage T7 ligase, and E. coli ligase. Thermostable ligases include, but are not limited to, Taq ligase, Tth ligase, and Pfu ligase. Thermostable ligase may be obtained from thermophilic or hyperthermophilic organisms, including but not limited to, prokaryotic, eukaryotic, or archael organisms. Enzymes appropriate for the ligation step are known in the art and include, e.g. T4 DNA ligase, Tth DNA ligase, Taq DNA ligase, Thermococcus sp. (strain 9°N) DNA ligase (9°N™ DNA ligase, New England Biolabs), and Ampligase™ (Epicentre Biotechnologies).

In this ligation step, a suitable ligase and any reagents that are necessary and/or desirable are combined with the reaction mixture and maintained under conditions sufficient for ligation of the junctions in the dsDNA complex to occur. As discussed further below, the ligation reaction may occur in a host cell, e.g. a bacterial cell. Ligation reaction conditions are well known to those of skill in the art. During ligation, the reaction mixture in certain embodiments may be maintained at a temperature ranging from about 4° C. to about 50° C., such as from about 20° C. to about 37° C. for a period of time ranging from about 5 seconds to about 16 hours, such as from about 1 minute to about 1 hour. In yet other embodiments, the reaction mixture may be maintained at a temperature ranging from about 35° C. to about 75° C., such as from about 37° C. to about 75° C., e.g., at or about 40° C., 50° C., 60° C. 70° C. or 75° C., for a period of time ranging from about 5 seconds to about 16 hours, such as from about 1 minute to about 1 hour, including from about 2 minutes to about 8 hours. In a representative example, the ligation reaction mixture includes 50 mM Tris pH7.5, 10 mM MgCl₂, 10 mM DTT, 1 mM ATP, 25 mg/ml BSA, and T4 DNA ligase at 0.125 units/ml. In yet another representative example, 2.125 mM magnesium ion, and 0.125 units/ml DNA ligase are employed.

As noted above, the ligation step typically results in the synthesis of a linear dsDNA molecule. However, when the dsDNA complex is contacted with a suitable linearised nucleic acid vector (e.g. plasmid) prior to ligation, the ligation step may result in the synthesis of a circular dsDNA molecule comprising the desired or target dsDNA molecule

The dsDNA molecule obtained from the method and use provided herein provides a further aspect of the invention.

It will be understood that the dsDNA molecule may be subjected to further processing steps following its synthesis, e.g. to isolate, modify and/or amplify the dsDNA molecule.

The ligation step of the method and use provided herein may not be completely efficient, i.e. there may be some unreacted (unligated) ssDNA molecules and/or undesired by-products. Thus, it may be desirable to separate the dsDNA molecule from unreacted components and/or other components in the reaction mixture, e.g. ligase. Any suitable means for separating the dsDNA molecule from other components in the reaction mixture may be employed.

Thus, the method may comprise a further step of separating or purifying dsDNA molecule. For example, the products of the ligation reaction may be separated by size using gel electrophoresis using an agarose gel or a polyacrylamide gel. The desired dsDNA molecule can then be isolated from the gel and purified further, if necessary, according to methods known in the art. Other methods for purifying, isolating or separating the dsDNA molecule utilise chromatography (e.g. HPLC, size-exclusion, ion-exchange, affinity, hydrophobic interaction, reverse-phase) or capillary electrophoresis.

As mentioned above, the dsDNA molecule produced by the method described above may contain a reactive group that is capable of reacting with another chemical group, e.g. a chemical group on a molecule or component to be conjugated to the dsDNA molecule, e.g. via click chemistry. For instance, conjugating additional molecules or components (which themselves may comprise or be viewed as functional groups) to the dsDNA molecule may be particularly useful for incorporating large or bulky groups, such as groups that may inhibit or lower the efficiency of the present method if present in the ssDNA molecules.

Accordingly, the method may comprise a step of conjugating a molecule or component to the dsDNA molecule via a functional (e.g. reactive) group in the dsDNA molecule, such as via click chemistry.

Alternatively viewed, the method may comprise a step of modifying the dsDNA molecule, e.g. to include a functional group, such as a label.

Thus, the method and use provided herein may result in a mixture of dsDNA molecules, i.e. a library of dsDNA molecules comprising a plurality of different (e.g. functionalised or modified) dsDNA molecules.

As noted above, a mixture of dsDNA molecules may be achieved by providing a plurality of versions (types) of one or more of the ssDNA molecules. The versions (types) of ssDNA molecules may have the same sequence and differ with respect to the nucleotides that contain a functional group (e.g. in the location and/or number of a functional group). Additionally or alternatively, the versions (types) of ssDNA molecules may have the same sequence and differ with respect to the type of functional groups on the modified nucleotides. For instance, each version (type) of a ssDNA molecule may contain a plurality of functional groups.

It also will be evident to the skilled person that a mixture of dsDNA molecules may be achieved by providing a plurality of versions (types) of one or more of the ssDNA molecules, wherein the versions (types) of ssDNA molecules may have different sequences (i.e. the versions may be sequence variants). Such sequence variants may be achieved using any suitable means known in the art. For instance, site-specific variants may be produced separately and mixed to provide the plurality of versions (types) of a ssDNA molecule. In a further representative example, variations in the sequence may be introduced randomly when enzymatically synthesising the ssDNA molecule, e.g. by performing the synthesis reaction at high salt concentrations or in the presence of Manganese ions to induce mutagenesis. The product of the synthesis reaction will be a mixture of ssDNA molecules comprising a plurality of versions of a ssDNA molecule (i.e. sequence variants of a ssDNA molecule). As noted above, when the plurality of versions of a ssDNA molecule are sequence variants each ssDNA molecule in the plurality will contain a common region of complementarity.

Alternatively, a mixture of dsDNA molecules may be achieved by performing the extension reaction at high salt concentrations or in the presence of Manganese ions to introduce mutations in the synthesised strand. The mutations may be introduced in the opposite strand in subsequent processing steps.

The method may also comprise a step of amplifying the dsDNA molecule. Methods for amplifying dsDNA molecules are well-known in the art and any suitable method may be employed. However, as noted above, PCR-based methods may result in the incorporation of errors (mutations) in the dsDNA products. Accordingly, it may be preferred to amplify the dsDNA molecule using methods other than PCR-based methods. Alternatively, if PCR-based methods are used to amplify the dsDNA molecule, it is preferred that the method uses a high-fidelity polymerase as defined above.

The method may involve amplifying the dsDNA using a host cell. For instance, the method may comprise:
(i) inserting the dsDNA molecule into a nucleic acid vector (e.g. a DNA plasmid);
(ii) amplifying the vector (e.g. DNA plasmid);
(iii) excising the dsDNA molecule from the vector; and optionally
(iv) separating or purifying the dsDNA molecule, e.g. from the vector nucleic acid.

Step (ii) may comprise transfecting the vector (e.g. DNA plasmid) into bacteria and growing the bacteria.

Suitable plasmid sequences are well-known in the art. Notably, inserting the dsDNA into a nucleic acid vector and amplifying the vector allows the sequence of the dsDNA to be checked (e.g. by sequencing) and for any errors in the sequence to be corrected, e.g. via iterations of sequencing and mutagenesis using any suitable methods. Thus, the method may comprise a step of checking the sequence of the dsDNA molecule, e.g. after step (i), (ii) or (iii) above, e.g. sequencing the dsDNA molecule. The method may also comprise a step of correcting one or more errors in the sequence of the dsDNA molecule, whereupon the corrected dsDNA may be amplified, e.g. via the method described above, i.e. steps (ii)-(iv) above.

The insertion of the dsDNA molecule into a plasmid also facilitates the generation of a significant number of copies of the dsDNA molecule. For instance, a single bacterial colony containing a plasmid comprising the sequence-verified dsDNA molecule may be grown to amplify the plasmid, which may be subsequently purified from the bacteria using any suitable means known in the art.

The dsDNA molecule may be excised from the plasmid, e.g. using restriction enzymes as described above. The excised linear dsDNA molecule may be purified using PAGE and gel extraction, or other suitable methods. The purified dsDNA molecule can then be used as desired.

In addition to amplification, the process of transfecting the dsDNA molecule containing plasmid into bacteria also allows a bacterial glycerol stock to be produced. Bacteria comprising the desired dsDNA molecule plasmid can be prepared in glycerol, frozen and stored stably for long periods of time.

The inventors have found that the dsDNA complex formed in the hybridisation step is very stable. Thus, where is it desirable to insert the DNA molecule into a nucleic acid vector (e.g. plasmid), the ligation step and insertion step advantageously may be performed in a single reaction. Thus, the ligation step may involve contacting (mixing) the dsDNA complex with a nucleic acid vector (e.g. a linearised plasmid) and means for ligating the polynucleotides (e.g. a ligase) under conditions suitable to form the dsDNA molecule and ligate it into the nucleic acid vector. This may step may have further advantages insofar as any single-stranded breaks in the vector (i.e. nicks) will be repaired when the vector is transferred into the host cell.

It will be understood by a person of skill in the art that the ligation step may be achieved or performed in a host cell (see e.g. Examples 1, 6 and 7). In this respect, the ligation step may involve contacting (mixing) the dsDNA complex with a nucleic acid vector (e.g. a linearised plasmid) and a suitable host cell (e.g. bacterial cell, such as E. coli) under conditions suitable for the host cell to take-up the assembled dsDNA complex: nucleic acid vector, wherein the ligase activity in the host cell will ligate the molecules to form a complete nucleic acid vector comprising the target dsDNA molecule. Alternatively viewed, the host cell will repair the single-stranded breaks (i.e. nicks) in the dsDNA complex and vector when the assembled dsDNA complex: nucleic acid vector is transferred into the host cell.

It will be evident that any suitable host cell may be used, e.g. a host cell comprising an enzyme with ligase activity, such as a ligase enzyme. In a representative example, the host cell is a bacterial host cell, such as an E. coli cell.

The stability of the dsDNA complex also facilitates the use of a thermostable polymerase to perform the extension step following assembly of the dsDNA complex, i.e. the reaction mixture containing the assembled dsDNA complex may be contacted with a polymerase (e.g. a thermostable polymerase) as described above and heated to a temperature that is optimal for the polymerase activity of the enzyme, e.g. a temperature that is higher than the temperature to which the reaction mixture was cooled during the hybridisation step.

Thus, in a representative example, provided herein is a method for producing a double stranded DNA (dsDNA) molecule comprising at least 3000 bp (e.g. at least 4000 bp or 5000 bp), the method comprising:
(a) providing:
(i) at least three (e.g. at least four or five) ssDNA molecules each comprising (e.g. consisting of) a non-overlapping portion of a first strand of the dsDNA molecule, wherein a first ssDNA molecule comprises the 5′ end of the first strand of the dsDNA molecule and a second ssDNA comprises the 3′ end of the first strand of the dsDNA molecule; and
(ii) at least four (e.g. at least five or six) ssDNA molecules each comprising (e.g. consisting of) a non-overlapping portion of a second strand of the dsDNA molecule, wherein: a first ssDNA molecule comprises the 5′ end of the second strand of the dsDNA molecule and is fully complementary a region of the second ssDNA molecule of (i); and a second ssDNA molecule comprises the 3′ end of the second strand of the dsDNA molecule and is fully complementary a region of the first ssDNA molecule of (i),
wherein:
(1) at least two (e.g. at least three or four) of the at least four (e.g. at least five or six) ssDNA molecules from (ii) that do not comprise the 5′ and 3′ ends of the second strand of the dsDNA molecule each comprise a first region that is fully complementary to an end region of a ssDNA molecule of (i) and a second region that is fully complementary to an end region of a different ssDNA molecule of (i);
(2) the ssDNA molecules of (i) to which the ssDNA molecules of (ii) are complementary form directly adjacent portions of the first strand of the dsDNA molecule;
(3) the regions that are complementary comprise at least 150 (e.g. at least 200 or 250) nucleotides;
(4) all of the ssDNA molecules comprise at least 400 (e.g. at least 500) nucleotides, optionally wherein all of the ssDNA molecules from (i) and the ssDNA molecules from (ii) that do not comprise the 5′ and 3′ ends of the second strand of the dsDNA molecule comprise at least 800 (e.g. at least 900 or 1000) nucleotides; and
(5) the ssDNA molecules are provided in equimolar amounts;
(b) hybridizing the ssDNA molecules that contain regions of complementarity to produce a fully dsDNA complex comprising all of the ssDNA molecules from (a); and
(c) ligating directly adjacent ssDNA molecules in the fully dsDNA complex to produce the dsDNA molecule.

In a further representative example provided herein is a method for producing a double stranded DNA (dsDNA) molecule comprising at least 3000 bp (e.g. at least 4000 bp or 5000 bp), the method comprising:
(a) providing:
(i) at least three (e.g. at least four or five) ssDNA molecules each comprising (e.g. consisting of) a non-overlapping portion of a first strand of the dsDNA molecule, wherein: a first ssDNA molecule comprises the 5′ end of the first strand of the dsDNA molecule; and a second ssDNA molecule comprises the 3′ end of the first strand of the dsDNA molecule; and
(ii) at least four (e.g. at least five or six) ssDNA molecules each comprising (e.g. consisting of) a non-overlapping portion of a second strand of the dsDNA molecule, wherein: a first ssDNA molecule comprises the 5′ end of the second strand of the dsDNA molecule and is fully complementary a region of the second ssDNA molecule of (i); and a second ssDNA molecule comprises the 3′ end of the second strand of the dsDNA molecule and is fully complementary a region of the first ssDNA molecule of (i),
wherein:
(1) at least two (e.g. at least three or four) of the at least four (e.g. at least five or six) ssDNA molecules from (ii) that do not comprise the 5′ and 3′ ends of the second strand of the dsDNA molecule each comprise a first region that is fully complementary to an end region of a ssDNA molecule of (i) and a second region that is fully complementary to an end region of a different ssDNA molecule of (i);
(2) the ssDNA molecules of (i) to which the ssDNA molecules of (ii) are complementary form indirectly adjacent portions of the first strand of the dsDNA molecule and the ssDNA molecules of (ii) to which the ssDNA molecules of (i) are complementary form indirectly adjacent portions of the second strand of the dsDNA molecule;
(3) the regions that are complementary comprise at least 150 (e.g. at least 200 or 250) nucleotides;
(4) all of the ssDNA molecules comprise at least 400 (e.g. at least 500) nucleotides, optionally wherein all of the ssDNA molecules from (i) and the ssDNA molecules from (ii) that do not comprise the 5′ and 3′ ends of the second strand of the dsDNA molecule comprise at least 800 (e.g. at least 900 or 1000) nucleotides; and
(5) the ssDNA molecules are provided in equimolar amounts;
(b) hybridizing the ssDNA molecules that contain regions of complementarity to produce a partially dsDNA complex comprising all of the ssDNA molecules from (a);
(c) extending the 3′ ends of the ssDNA molecules in the partially dsDNA complex to produce a fully dsDNA complex; and
(d) ligating directly adjacent ssDNA molecules in the fully dsDNA complex to produce the dsDNA molecule.

In yet further representative example provided herein is a method for producing a double stranded DNA (dsDNA) molecule comprising at least 3000 bp (e.g. at least 4000 bp or 5000 bp), the method comprising:
(a) providing:
(i) at least three (e.g. at least four or five) ssDNA molecules each comprising (e.g. consisting of) a non-overlapping portion of a first strand of the dsDNA molecule, wherein: a first ssDNA molecule comprises the 5′ end of the first strand of the dsDNA molecule; and a second ssDNA molecule comprises the 3′ end of the first strand of the dsDNA molecule; and
(ii) at least three (e.g. at least four or five) ssDNA molecules each comprising (e.g. consisting of) a non-overlapping portion of a second strand of the dsDNA molecule, wherein: a first ssDNA molecule comprises the 5′ end of the second strand of the dsDNA molecule and is fully complementary a region of the second ssDNA molecule of (i),
wherein:
(1) at least two (e.g. at least three or four) of the at least three (e.g. at least four or five) ssDNA molecules from (ii) that do not comprise the 5′ end of the second strand of the dsDNA molecule each comprise a first region that is fully complementary to an end region of a ssDNA molecule of (i) and a second region that is fully complementary to an end region of a different ssDNA molecule of (i);
(2) the ssDNA molecules of (i) to which the ssDNA molecules of (ii) are complementary form directly adjacent portions of the first strand of the dsDNA molecule and the ssDNA molecules of (ii) to which the ssDNA molecules of (i) are complementary form indirectly adjacent portions of the second strand of the dsDNA molecule;
(3) the regions that are complementary comprise at least 50 (e.g. at least 75 or 100) nucleotides;
(4) all of the ssDNA molecules of (i) comprise at least 800 (e.g. at least 900 or 1000) nucleotides and all of the ssDNA molecules of (ii) comprise at least 150 (e.g. at least 175 or 200) nucleotides; and
(5) the ssDNA molecules are provided in equimolar amounts;
(b) hybridizing the ssDNA molecules that contain regions of complementarity to produce a partially dsDNA complex comprising all of the ssDNA molecules from (a);
(c) extending the 3′ ends of the ssDNA molecules of (ii) in the partially dsDNA complex to produce a fully dsDNA complex; and
(d) ligating directly adjacent ssDNA molecules in the fully dsDNA complex to produce the dsDNA molecule.

The invention will now be described in more detail in the following non-limiting Examples with reference to the following drawings:

FIG. 1 shows a schematic of the assembly strategies: (A) complete overlapping; (B) partial overlapping; and (C) minimal overlapping, wherein ssDNA molecules labelled A-P refer to SEQ ID NOs: 1-16, respectively.

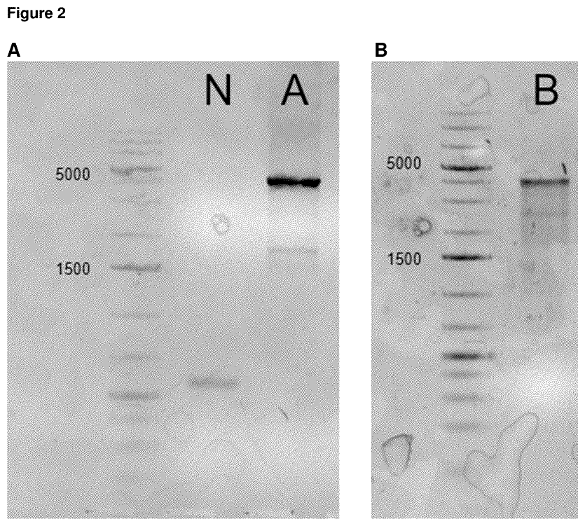

FIG. 2 shows photographs of agarose gels stained with ethidium bromide and visualised with a Bio-Rad Chemidoc MP, wherein: (A) shows the product of the complete overlapping reaction described in Example 1 in lane A and N shows a negative control reaction; and (B) shows the product of the partial overlapping reaction described in Example 1 in lane B.

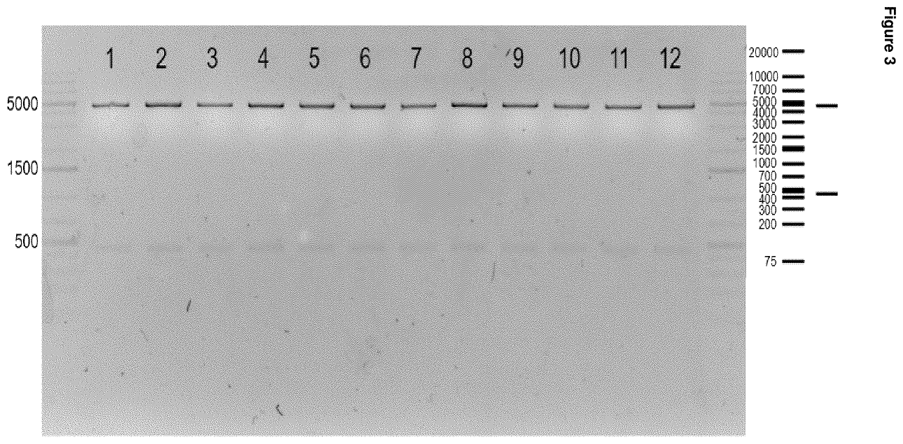

FIG. 3 shows photographs of agarose gels stained with ethidium bromide and visualised with a Bio-Rad Chemidoc MP, wherein: lanes 1-6 show the digestion products plasmids containing products from the partial overlapping reaction described in Example 2; and lanes 7-12 show the digestion products plasmids containing products from the minimal overlapping reaction described in Example 1.

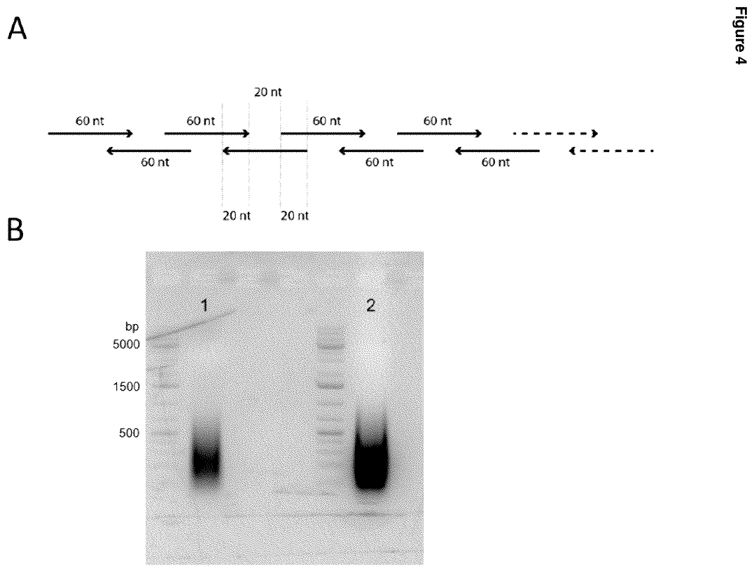

FIG. 4 shows comparative assembly strategy of using shorter ssDNA molecules to produce dsDNA, wherein (A) shows a schematic of the ssDNA oligonucleotides and their annealing pattern; and (B) shows a photograph of an agarose gel stained with ethidium bromide and visualised with a Bio-Rad Chemidoc MP to measure final gene assembly products, wherein lane 1 shows 100 ng of the assembly mix loaded and lane 2 shows 500 ng of the assembly mix loaded (ladder: Gene ruler 1 kb Plus).

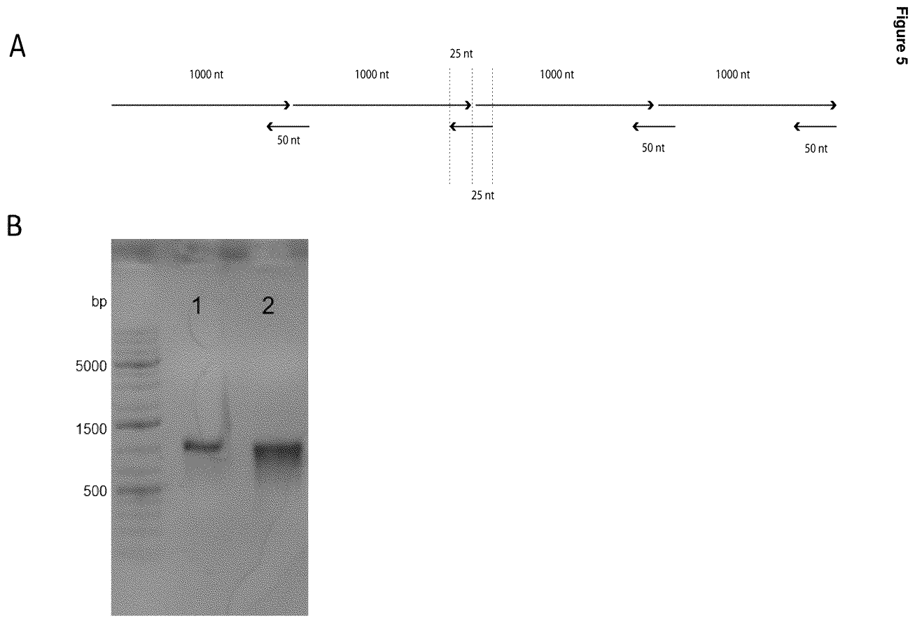

FIG. 5 shows the minimal overlapping approach with oligonucleotides containing short regions of complementarity, wherein (A) shows a schematic of the assembly using portions A, B, C and D and ssDNA connectors (each 50 nt long); and (B) shows a photograph of an agarose gel stained with ethidium bromide and visualised with a Bio-Rad Chemidoc MP to observe final gene assembly products, wherein lane 1 shows 50 ng of the assembly mix loaded and lane 2 shows 100 ng of the assembly mix loaded (ladder: Gene ruler 1 kb Plus).

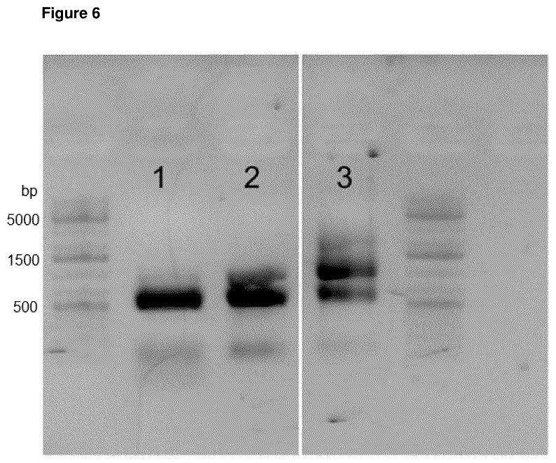

FIG. 6 shows a photograph of an agarose gel stained with ethidium bromide and visualised with a Bio-Rad Chemidoc MP to visualise the RCA digestion products obtained using different concentrations of magnesium and manganese (samples 1-3 are visualised in lanes 1-3, respectively; see Table 1 for sample components).

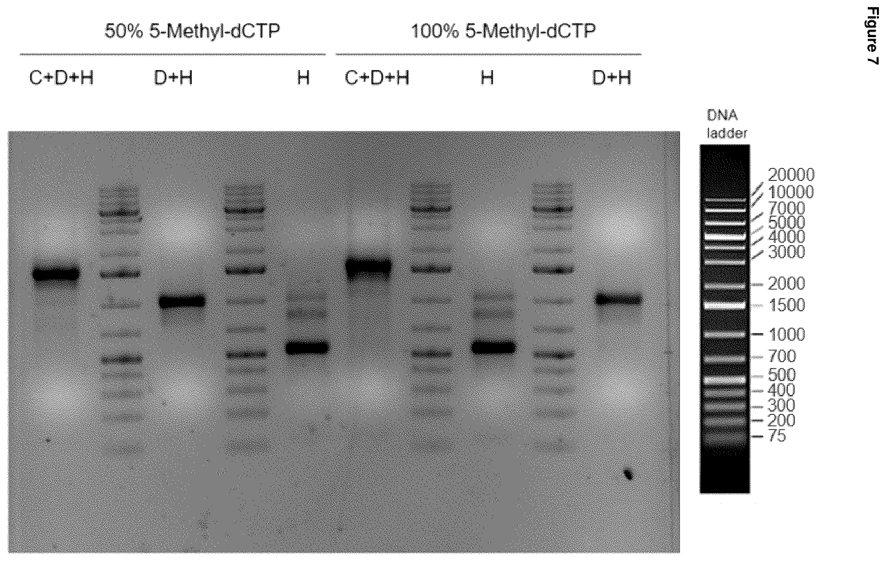

FIG. 7 shows a photograph of an agarose gel stained with ethidium bromide and visualised with a Bio-Rad Chemidoc MP to observe the assembly of segments C, D and H containing 50% or 100% of 5-Methyl-dCTP.

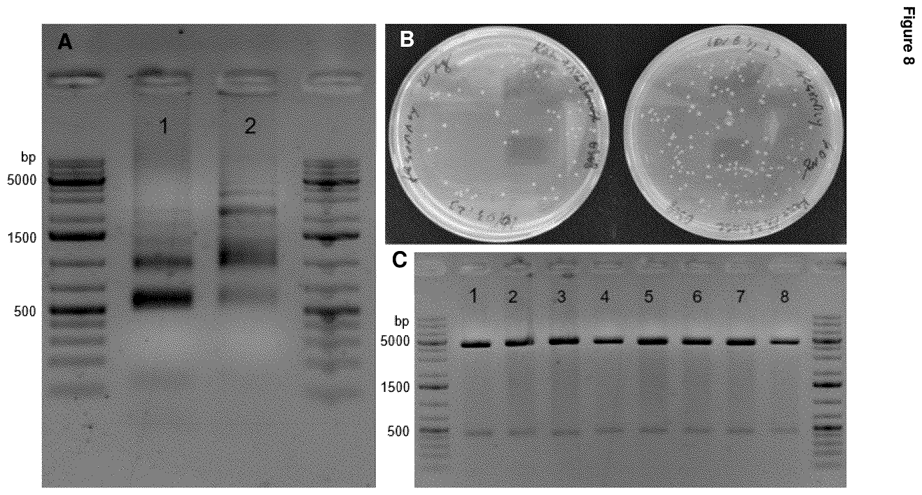

FIG. 8 shows the results of the experiments described in Example 6, wherein: (A) shows a photograph of an agarose gel stained with ethidium bromide and visualised with a Bio-Rad Chemidoc MP to show the assembly from ssDNA molecules produced in two separate one-pot synthesis reactions (plus and minus ssDNA molecules) (lane 1:50 ng total DNA loaded; lane 2:100 ng total DNA loaded); (B) shows Escherichia coli colonies transformed with plasmids generated from ligation reactions with assembly mixes (kanamycin selection); and (C) shows a photograph of an agarose gel stained with ethidium bromide and visualised with a Bio-Rad Chemidoc MP showing the right digestion pattern for plasmids from eight colonies digested with Ndel (with two bands present in each lane corresponding to 4.8 kbp and 0.45 kbp).

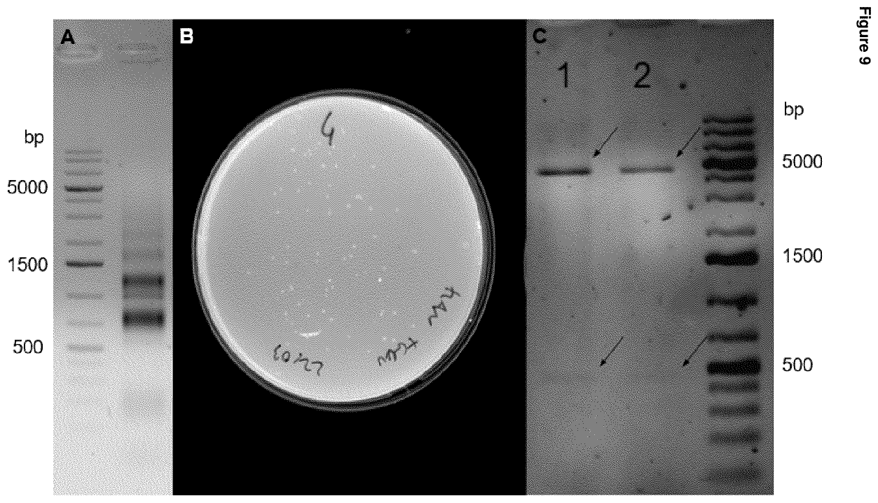

FIG. 9 demonstrates one-pot synthesis and assembly using the minimal overlapping approach described in Example 7, wherein: (A) shows a photograph of an agarose gel stained with ethidium bromide and visualised with a Bio-Rad Chemidoc MP to show the digestion products of a one-pot RCA containing all the necessary segments for the minimal overlapping strategy; (B) is a photograph of a plate containing kanamycin which shows Escherichia coli colonies transfected with plasmids generated from the ligation of a linearised plasmid and the dsDNA complex produced from hybridisation of the digestion products of the one-pot RCA; and (C) shows a photograph of an agarose gel stained with ethidium bromide and visualised with a Bio-Rad Chemidoc MP to demonstrate the digestion of plasmids using Ndel following their extraction from two single colonies.

### EXAMPLES

### Example 1: Production of dsDNA Using the Different Overlapping Strategies

A synthetic gene (4000 bp) comprising a nucleotide sequence encoding GFP operably linked to promoter for expression in mammalian cells, and a kanamycin/neomycin resistance cassette (SEQ ID NO: 17). Single stranded polynucleotides comprising non-overlapping portions (fragments) of a gene were designed according to different strategies (see FIG. 1) and enzymatically synthesised using the RCA-based MOSIC method.

### Strategies:

1) Complete overlapping: the plus strand of the gene was divided into four portions of 1000 nucleotides (nt) each (A, B, C, D); the minus strand was divided into three portions of 1000 and two portions of 500 nt (E, F, G, H, I). The two portions of 500 nucleotides (E, I) are placed at the two extremes, so that the 1000 nt long portions overlap with each other by 500 nucleotides.
2) Partial overlapping: The portions were designed to have overlaps of 250 nucleotides. The plus strand has three portions of 1000 nucleotides (A, J, D), where A and D are the same as the complete overlapping strategy, and J stretches from 1501 to 2500 nucleotides. The minus strand has the portions E and I at the two extremes, and two 1000 nt long portions (K, L) between them, so that the overlap among the 1000 portions is 250 bases long.
3) Minimal overlapping: the plus strand is identical to the complete overlapping strategy, and the portions A, B, C and D are used. The minus strand is made up of four 200 nt long portions: three of them connect the 1000 nt portions with an overlap of 100 bases (M, N, O), and the fourth anneals at the 5′ of the minus strand (P).

The ssDNA molecules (polynucleotides) were purified, mixed in stoichiometric quantities and heated to 95° C. for 10 minutes. The mix underwent a temperature ramp from 95° C. to room temperature. In case of the partial and minimal overlapping strategies, the gaps between the assembled fragments were filled-in by polymerase-mediated extension using a high-fidelity thermostable polymerase (Q5@ High-Fidelity DNA Polymerase (NEB)). The assembled fragments (see FIG. 2) were ligated and inserted in a bacterial plasmid, either in separate ligation steps (using T4 DNA ligase) or in a single step (using Taq DNA ligase).

Escherichia coli DH5alpha competent cells were transformed with the final ligated plasmid. The cells were selected on kanamycin and plasmids were extracted from single colonies and tested first by restriction endonuclease digestion (FIG. 3), and then by Sanger sequencing, which verified that the synthesised and amplified dsDNA molecules contain the correct nucleotide sequences.

### Example 2 (Comparative): Attempted Production of dsDNA Using a One-Pot Assembly with Short ssDNA Molecules

To demonstrate that long ssDNA molecules with long regions of complementarity are essential for the disclosed overlapping strategies to work, a comparative experiment was performed using a one-pot assembly method with short oligonucleotides (SEQ ID NOs: 18-117). Each oligonucleotide is 60 nt long, with the exception of SEQ ID NO: 68, which is only 40 nt long akin to segment I in FIG. 1B. Moreover, an oligonucleotide containing the 3′ end of the second strand (corresponding to segment E in FIG. 1B) was omitted on the basis that the penultimate oligonucleotide (corresponding to segment K in FIG. 1B) may be extended using the most 5′ oligonucleotide of the first strand (corresponding to segment A in FIG. 1B) to provide the 3′ end of the second strand of the desired dsDNA molecule. Similarly, the most 3′ oligonucleotide of the first strand (corresponding to segment D in FIG. 1B) does not contain the 3′ end of the desired dsDNA molecule on the basis that the oligonucleotide that provides the 5′ end of the second strand of the desired dsDNA may template the extension of the most 3′ oligonucleotide of the first strand to provide the 3′ end of the first strand of the desired dsDNA molecule.

Each oligonucleotide has at least one region of complementarity (overlap) of 20 bases to an oligonucleotide forming the opposite strand. The arrangement of partially overlapping oligonucleotides is repeated along the entire length of the dsDNA to be produced (see FIG. 4A), i.e. a partial overlapping strategy as defined herein was used.

Two different concentrations of oligonucleotides were tested: 200 ng and 1000 ng. Oligonucleotides were mixed in a PCR buffer and incubated using a temperature ramp (95° C. for 5 min, followed by a temperature ramp down at a rate of 0.1° C./s to 22° C. for 1 hr). The gaps between the assembled fragments were filled-in by polymerase-mediated extension using a high-fidelity thermostable polymerase (Q5® High-Fidelity DNA Polymerase (NEB)) together with 1 mM nucleotide during a 15 min incubation at 72° C., as suggested by the manufacturer.

Subsequently, half of the reaction samples were loaded on an agarose gel following which gel electrophoresis and ethidium bromide staining were performed. No bands corresponding to assembly products (i.e. dsDNA complexes) were observed on the gel (see FIG. 4B). No colonies were obtained when the assembly mixes were combined with a linearised plasmid for the ligase-mediated production of plasmids containing the desired dsDNA molecule via their subsequent transformation of Escherichia coli strains.

These results indicate that short oligonucleotides, i.e. containing less than 150 nt, with short overlaps, i.e. less than 50 nt, cannot be used in the methods disclosed herein to produce long dsDNA molecules, i.e. dsDNA molecules comprising at least 1500 bp.

### Example 3 (Comparative): Use of the Minimal Overlapping Strategy with Oligonucleotides Having Short Regions of Complementarity (Overlaps)

For this comparative experiment it was attempted produce a dsDNA molecule using ssDNA molecules A, B, C and D (as described in Example 1) to form the first strand and short oligonucleotides (50 nt long, SEQ ID NOs: 118-121) to form the second strand via a minimal overlapping strategy as defined herein. The short oligonucleotides have regions of complementary of 25 nt to adjacent ssDNA molecules in the first strand, with the exception of the oligonucleotide that forms the 5′ end of the second strand (corresponding to segment P in FIG. 1C), which is fully complementary to segment D (see FIG. 5A).

Assembly was attempted with two different concentrations of DNA (sample 1=100 ng and sample 2=200 ng). Segments were mixed in a PCR buffer and incubated using a temperature ramp (95° C. for 5 min, followed by a temperature ramp down at a rate of 0.1° C./s to 22° C. for 1 hr). Given the use of the minimal overlapping strategy, the gaps between the assembled fragments were filled-in by polymerase-mediated extension using a high-fidelity thermostable polymerase (Q5® High-Fidelity DNA Polymerase (NEB)) together with 1 mM nucleotide during a 15 min incubation at 72° C.

Half of the reaction samples were loaded on an agarose gel following which gel electrophoresis and ethidium bromide staining were performed. No bands corresponding to assembly products (dsDNA complexes) were subsequently observed on the gel (FIG. 5B).

### Example 4: Use of Single Long Oligonucleotide Mutations for the Assembly of a Degenerate DNA Library

As described elsewhere herein, the methods disclosed herein enable the assembly of degenerate DNA libraries, wherein the degenerate region can be site specific for long DNA sequences. To demonstrate this, a degenerate ssDNA (1 kb long) was synthesised using the MOSIC method described herein for assembly in a desired dsDNA molecule.

Three RCA reactions using phi29 DNA polymerase, 1 mM dNTPs, and with different concentrations of magnesium and manganese (see Table 1 for the concentrations used in each reaction) were run for 16 hrs at 30° C. using the circular nicked dsDNA template which encodes for segment B described in Example 1. After inactivation at 70° C., the RCA products were digested using the BtsCI restriction enzyme (to remove the hairpins and release the ssDNA products). Digestion products, after heat inactivation at 80° C., were run on an agarose gel (see FIG. 6) and analyzed by Illumina sequencing.

Sequencing showed that the error rate increased as the concentration of manganese increased.

The degenerate ssDNA molecules produced using this method may be used in the methods disclosed herein to produce libraries of degenerate dsDNA molecules.

TABLE 1
error rates measured according to the concentration
of magnesium and manganese used in each reaction

| Samples | 1 | 2 | 3 |
|---|---|---|---|
| MgCl₂ | 5 mM | 5 mM | / |
| MnCl₂ | 1.5 mM | 3 mM | 10 mM |
| Error rate (Illumina) | 1.59 × 10⁻³ | 2.61 × 10⁻³ | 2.68 × 10⁻³ |

### Example 5: Assembly of ssDNA Molecules Containing Modified Nucleotides

The methods disclosed herein enable the assembly of dsDNA molecules containing site-specific modifications. To demonstrate this, single modified segments (ssDNA molecules) were produced as previously described (WO2020/161187); the chosen modification was 5-Methyl-2′-deoxycytidine-5′-triphosphate (5-Methyl-dCTP).

Using the RCA and BtsCI digestion protocols described in Example 4, segments C, D and H were synthesised in single tubes. Each segment was produced using two different RCA conditions, with either 50% or 100% of the natural dCTP replaced by 5-Methyl-dCTP.

After desalting the produced ssDNA molecules using Nucleospin, segments were assembled in different combinations using 50 ng of each segment in each reaction as follows:
1) Segments C, D and H containing 50% of 5-Methyl-dCTP;
2) Segments D and H containing 50% of 5-Methyl-dCTP;
3) Segments C, D and H containing 100% of 5-Methyl-dCTP;
4) Segments D and H containing 100% of 5-Methyl-dCTP.

Segments were subsequently mixed in a PCR buffer and incubated using a temperature ramp (95° C. for 5 min, followed by a temperature ramp down at a rate of 0.1° C./s to 22° C. for 1 hr) to form dsDNA complexes.

Samples were then loaded on an agarose gel following which gel electrophoresis and ethidium bromide staining were performed. A negative control (portion H only) was also loaded into the gel.

Bands corresponding to the assembly of the segments C, D and H (i.e. dsDNA complexes comprising the segments C, D and H) containing 50% or 100% of 5-Methyl-dCTP were observed on the gel (see FIG. 7).

This demonstrates that ssDNA molecules comprising modified nucleotides may be used in the methods disclosed herein to produce dsDNA molecules containing site-specific modifications.

### Example 6: Separate Synthesis of ssDNA Molecules Forming the Plus and Minus Strands and One-Pot Assembly of a Desired dsDNA Molecule

The ssDNA molecules that form the plus and minus strands for the assembly of a dsDNA molecule using in the methods disclosed herein may be synthesised separately and subsequently assembled into the dsDNA molecule via one-pot assembly. It would be advantageous to minimise the number of synthesis reactions required to produce the ssDNA molecules as this would simplify the assembly process.

To investigate whether it is possible to reduce the number of synthesis steps, a desired dsDNA molecule was synthesised using the minimal overlapping strategy, wherein ssDNA molecules for each strand were synthesised together, i.e. in synthesis pools. The MOSIC method described herein was used to synthesise the ssDNA molecules.

Segments A, B, C and D were produced in a single tube, mixing four circular nicked templates each encoding for a single segment (i.e., segments A-D were produced from separate circular templates). The segments M, N, O and P were produced in a second one-pot RCA reaction, with one circular template encoding for M, N, O and P.

In both cases, RCAs were performed using phi29 DNA polymerase at 30° C. for 16 hrs, and the RCA products were digested using BtsCI at 50° C. for 4 hrs to release the ssDNA segments from the cutter hairpins. After incubating at 70° C. for 15 min to inactivate the polymerase and desalting the digestion mix with Nucleospin, the ssDNA pools were used in two different quantities (sample 1=100 ng and sample 2=200 ng).

The segments for both strands were resuspended in a PCR buffer in a single tube and incubated using a temperature ramp (95° C. for 5 min, followed by a temperature ramp down at a rate of 0.1° C./s to 22° C. for 1 hr) to form the dsDNA complexes. Given the use of the minimal overlapping strategy, the gaps between the assembled segments were filled-in by polymerase-mediated extension using a high-fidelity thermostable polymerase (Q5@ High-Fidelity DNA Polymerase (NEB)) together with 1 mM nucleotide during a 15 min incubation at 72° C., as suggested by the manufacturer.

Half of the sample reactions were loaded on an agarose gel following which gel electrophoresis and ethidium bromide staining were performed. Bands on the gel show that different states of assembly were visible, i.e. various dsDNA complexes were formed (see FIG. 8A). Both assembly mixes (samples 1 and 2) were mixed with a linearised plasmid and transformed into Escherichia coli, and many colonies grew overnight (see FIG. 8B), demonstrating that the ligation step occurred within the bacterial cells. Eight random colonies were selected from which plasmids were extracted and digested with Ndel to verify the presence of complete inserts.

All digestion mixtures resulted in a digestion pattern on the agarose gel corresponding to the fully assembled dsDNA molecule (FIG. 8C). Sanger sequencing showed a 100% correct sequence for each insert was derived from the selected colonies.

### Example 7: One-Pot Synthesis and Assembly Using the Minimal Overlapping Strategy

A one-pot method for the synthesis and assembly of long ssDNA molecules would be particularly advantageous as this would further simplify the method relative to Example 6, which involved separate synthesis of the ssDNA molecules for each strand. The possibility of using one-pot assembly and synthesis was investigated using the minimal overlapping strategy.

All ssDNA molecules (segments A, B, C, D, M, N, O and P) were synthesised as described in Example 6 but in a single tube. RCA was performed by incubating the four circular nicked dsDNA encoding for segments A, B, C and D, and the circular nicked dsDNA template encoding for segments M, N, O and P, together with phi29 DNA polymerase and dNTPs for 16 hrs at 30° C.

After heat inactivation, RCA products were digested as previously described using BtsCI to obtain the single ssDNA molecules. Even after digestion it was possible to observe the partial assembly of the sequences using an agarose gel following which gel electrophoresis and ethidium bromide staining were performed. (FIG. 9A).

After desalting with Nucleospin, the digestion mix was resuspended in a PCR buffer and incubated using a temperature ramp for assembly (95° C. for 5 min, followed by a temperature ramp down at a rate of 0.1° C./s to 22° C. for 1 hr) to form the dsDNA complexes. Due to the use of the minimal overlapping strategy, the gaps between the assembled fragments were filled-in by polymerase-mediated extension using a high-fidelity thermostable polymerase (Q5@ High-Fidelity DNA Polymerase (NEB)) together with 1 mM nucleotide during a 15 min incubation at 72° C., as suggested by the manufacturer. Other polymerases such as Phusion DNA polymerase or Taq polymerase were tested, giving the same outcome.

The resulting dsDNA complexes were mixed with a linearised plasmid and transformed into Escherichia coli, and many colonies grew on the selection plate (FIG. 9B), demonstrating that the ligation step occurred within the bacterial cells. Two colonies were selected, from which plasmids were extracted and digested with Ndel. The digested plasmid samples were applied to an agarose gel following which gel electrophoresis and ethidium bromide staining were performed and showed that the plasmids generated the correct bands (0.45 kbp and 4.8 kbp; top and bottom arrows respectively in FIG. 9C), confirming the assembly strategy had worked correctly. Sanger sequencing also confirmed that both inserts were 100% correct.

This experiment demonstrates that the methods disclosed herein advantageously and surprisingly may be performed in a one-pot reaction, comprising both the synthesis of the ssDNA molecules and their subsequent assembly into the desired dsDNA molecule.

1. A method for producing a double stranded DNA (dsDNA) molecule comprising at least 1500 bp, the method comprising:
(a) providing:
(i) a plurality of single stranded DNA (ssDNA) molecules each comprising (e.g. consisting of) a non-overlapping portion of a first strand of the dsDNA molecule, wherein a first ssDNA molecule comprises the 5′ end of the first strand of the dsDNA molecule and a second ssDNA: (a) comprises the 3′ end of the first strand of the dsDNA molecule or (b) is capable of providing the 3′ end of the first strand of the dsDNA molecule via a polymerase-mediated extension reaction using a ssDNA molecule comprising the 5′ end of the second strand as a template; and
(ii) a plurality of ssDNA molecules each comprising (e.g. consisting of) a non-overlapping portion of a second strand of the dsDNA molecule, wherein a first ssDNA molecule comprises the 5′ end of the second strand of the dsDNA molecule and, optionally, a second ssDNA comprises the 3′ end of the second strand of the dsDNA molecule,
wherein:
(1) at least two of the plurality of ssDNA molecules from (ii) each comprise a first region that is fully complementary to an end region of a ssDNA molecule of (i) and a second region that is fully complementary to an end region of a different ssDNA molecule of (i);
(2) the ssDNA molecules of (i) to which the ssDNA molecules of (ii) are complementary form adjacent portions of the first strand of the dsDNA molecule;
(3) the regions that are complementary comprise at least 50 nucleotides;
(4) all of the ssDNA molecules comprise at least 150 nucleotides; and
(5) the ssDNA molecules are provided in equimolar amounts;
(b) hybridizing the ssDNA molecules that contain regions of complementarity to produce a dsDNA complex comprising all of the ssDNA molecules from (a); and
(c) ligating adjacent ssDNA molecules using a ligase enzyme to produce the dsDNA molecule.

2. The method for producing a double stranded DNA (dsDNA) molecule according to claim 1, the method comprising:
(a) providing:
(i) at least three ssDNA molecules each comprising (e.g. consisting of) a non-overlapping portion of a first strand of the dsDNA molecule, wherein a first ssDNA molecule comprises the 5′ end of the first strand of the dsDNA molecule and a second ssDNA comprises the 3′ end of the first strand of the dsDNA molecule; and
(ii) at least four ssDNA molecules each comprising (e.g. consisting of) a non-overlapping portion of a second strand of the dsDNA molecule, wherein: a first ssDNA molecule comprises the 5′ end of the second strand of the dsDNA molecule and is fully complementary a region of the second ssDNA molecule of (i); and a second ssDNA molecule comprises the 3′ end of the second strand of the dsDNA molecule and is fully complementary a region of the first ssDNA molecule of (i),
wherein:
(1) at least two of the at least four ssDNA molecules from (ii) that do not comprise the 5′ and 3′ ends of the second strand of the dsDNA molecule each comprise a first region that is fully complementary to an end region of a ssDNA molecule of (i) and a second region that is fully complementary to an end region of a different ssDNA molecule of (i);
(2) the ssDNA molecules of (i) to which the ssDNA molecules of (ii) are complementary form directly adjacent portions of the first strand of the dsDNA molecule;
(3) the regions that are complementary comprise at least 50 nucleotides;
(4) all of the ssDNA molecules comprise at least 150 nucleotides; and
(5) the ssDNA molecules are provided in equimolar amounts;
(b) hybridizing the ssDNA molecules that contain regions of complementarity to produce a fully dsDNA complex comprising all of the ssDNA molecules from (a); and
(c) ligating directly adjacent ssDNA molecules using a ligase enzyme to produce the dsDNA molecule.

3. The method for producing a double stranded DNA (dsDNA) molecule according to claim 1, the method comprising:
(a) providing:
(i) at least three ssDNA molecules each comprising (e.g. consisting of) a non-overlapping portion of a first strand of the dsDNA molecule, wherein: a first ssDNA molecule comprises the 5′ end of the first strand of the dsDNA molecule; and a second ssDNA molecule comprises the 3′ end of the first strand of the dsDNA molecule; and
(ii) at least four ssDNA molecules each comprising (e.g. consisting of) a non-overlapping portion of a second strand of the dsDNA molecule, wherein: a first ssDNA molecule comprises the 5′ end of the second strand of the dsDNA molecule and is fully complementary a region of the second ssDNA molecule of (i); and a second ssDNA molecule comprises the 3′ end of the second strand of the dsDNA molecule and is fully complementary a region of the first ssDNA molecule of (i),
wherein:
(1) at least two of the at least four ssDNA molecules from (ii) that do not comprise the 5′ and 3′ ends of the second strand of the dsDNA molecule each comprise a first region that is fully complementary to an end region of a ssDNA molecule of (i) and a second region that is fully complementary to an end region of a different ssDNA molecule of (i);
(2) the ssDNA molecules of (i) to which the ssDNA molecules of (ii) are complementary form indirectly adjacent portions of the first strand of the dsDNA molecule and the ssDNA molecules of (ii) to which the ssDNA molecules of (i) are complementary form indirectly adjacent portions of the second strand of the dsDNA molecule;
(3) the regions that are complementary comprise at least 50 nucleotides;
(4) all of the ssDNA molecules comprise at least 150 nucleotides; and
(5) the ssDNA molecules are provided in equimolar amounts;
(b) hybridizing the ssDNA molecules that contain regions of complementarity to produce a partially dsDNA complex comprising all of the ssDNA molecules from (a);
(c) extending the 3′ ends of the ssDNA molecules to produce a fully dsDNA complex; and
(d) ligating directly adjacent ssDNA molecules using a ligase enzyme to produce the dsDNA molecule.

4. The method for producing a double stranded DNA (dsDNA) molecule according to claim 1, the method comprising:
(a) providing:
(i) at least three ssDNA molecules each comprising (e.g. consisting of) a non-overlapping portion of a first strand of the dsDNA molecule, wherein: a first ssDNA molecule comprises the 5′ end of the first strand of the dsDNA molecule;
and a second ssDNA molecule comprises the 3′ end of the first strand of the dsDNA molecule; and
(ii) at least three ssDNA molecules each comprising (e.g. consisting of) a non-overlapping portion of a second strand of the dsDNA molecule, wherein: a first ssDNA molecule comprises the 5′ end of the second strand of the dsDNA molecule and is fully complementary a region of the second ssDNA molecule of (i),
wherein:
(1) at least two of the at least three ssDNA molecules from (ii) that do not comprise the 5′ end of the second strand of the dsDNA molecule each comprise a first region that is fully complementary to an end region of a ssDNA molecule of (i) and a second region that is fully complementary to an end region of a different ssDNA molecule of (i);
(2) the ssDNA molecules of (i) to which the ssDNA molecules of (ii) are complementary form directly adjacent portions of the first strand of the dsDNA molecule and the ssDNA molecules of (ii) to which the ssDNA molecules of (i) are complementary form indirectly adjacent portions of the second strand of the dsDNA molecule;
(3) the regions that are complementary comprise at least 50 nucleotides;
(4) all of the ssDNA molecules comprise at least 150 nucleotides; and
(5) the ssDNA molecules are provided in equimolar amounts;
(b) hybridizing the ssDNA molecules that contain regions of complementarity to produce a partially dsDNA complex comprising all of the ssDNA molecules from (a);
(c) extending the 3′ ends of the ssDNA molecules of (ii) to produce a fully dsDNA complex; and
(d) ligating directly adjacent ssDNA molecules using a ligase enzyme to produce the dsDNA molecule.

5. The method for producing a double stranded DNA (dsDNA) molecule according to claim 1, the method comprising:
(a) providing:
(i) at least three ssDNA molecules each comprising (e.g. consisting of) a non-overlapping portion of a first strand of the dsDNA molecule, wherein: a first ssDNA molecule comprises the 5′ end of the first strand of the dsDNA molecule; and a second ssDNA molecule: (a) comprises the 3′ end of the first strand of the dsDNA molecule or (b) is capable of providing the 3′ end of the first strand of the dsDNA molecule via a polymerase-mediated extension reaction using a ssDNA molecule comprising the 5′ end of the second strand as a template; and
(ii) at least three (e.g. at least four) ssDNA molecules each comprising (e.g. consisting of) a non-overlapping portion of a second strand of the dsDNA molecule, wherein: a first ssDNA molecule comprises the 5′ end of the second strand of the dsDNA molecule and is fully complementary a region of the second ssDNA of (i); and, optionally, a second ssDNA molecule comprises the 3′ end of the second strand of the dsDNA molecule and is fully complementary a region of the first ssDNA molecule of (i),
wherein:
(1) at least two of the at least three (e.g. at least two of the at least four) ssDNA molecules from (ii) that do not comprise the 5′ and 3′ ends of the second strand of the dsDNA molecule each comprise a first region that is fully complementary to an end region of a ssDNA molecule of (i) and a second region that is fully complementary to an end region of a different ssDNA molecule of (i);
(2) the ssDNA molecules of (i) to which the ssDNA molecules of (ii) are complementary form indirectly adjacent portions of the first strand of the dsDNA molecule and the ssDNA molecules of (ii) to which the ssDNA molecules of (i) are complementary form indirectly adjacent portions of the second strand of the dsDNA molecule;
(3) the regions that are complementary comprise at least 50 nucleotides;
(4) all of the ssDNA molecules comprise at least 150 nucleotides; and
(5) the ssDNA molecules are provided in equimolar amounts;
(b) hybridizing the ssDNA molecules that contain regions of complementarity to produce a partially dsDNA complex comprising all of the ssDNA molecules from (a);
(c) extending the 3′ ends of the ssDNA molecules to produce a fully dsDNA complex; and
(d) ligating directly adjacent ssDNA molecules using a ligase enzyme to produce the dsDNA molecule.

6. The method of any one of claims 1 to 5, wherein the ssDNA molecules of (a) (i) comprise at least 500 nucleotides, optionally at least 600, 700, 800, 900 or 1000 nucleotides.

7. The method of any one of claim 1 to 3 or 5, wherein: (A) the ssDNA molecules of (a) (i) comprise at least 500 nucleotides, optionally at least 600, 700, 800, 900 or 1000 nucleotides; and (B) at least two of the ssDNA molecules of (a) (ii) comprise at least 500 nucleotides, optionally at least 600, 700, 800, 900 or 1000 nucleotides.

8. The method of claim 1 or 4, wherein: (A) the ssDNA molecules of (a) (i) comprise at least 500 nucleotides, optionally at least 600, 700, 800, 900 or 1000 nucleotides; and (B) the ssDNA molecules of (a) (ii) are shorter than the ssDNA molecules of (a) (i).

9. The method of any one of claims 1 to 8, wherein the regions that are complementary comprise at least 100 nucleotides, optionally at least 150, 200, 250, 300, 400 or 500 nucleotides.

10. The method of any one of claims 1 to 9, wherein the dsDNA molecule comprises at least 2000 bp, 2500 bp, 3000 bp, 3500 bp or 4000 bp.

11. The method of any one of claims 1 to 9, wherein the dsDNA molecule comprises 1500-50000 bp.

12. The method of any one of claims 1 to 11, wherein the ssDNA molecules are produced using enzymatic synthesis.

13. The method of claim 12, wherein the enzymatic synthesis is asymmetric PCR, TdT synthesis, an RCA-based synthesis method or a combination thereof.

14. The method of any one of claims 1 to 13, wherein the one or more (preferably all) of the ssDNA molecules provided in (a) are produced using a method comprising:
(a) providing a circular DNA molecule comprising a nucleotide sequence encoding the ssDNA molecule bordered by cleavage domains;
(b) performing a rolling circle amplification (RCA) reaction with the circular DNA molecule of (a) as a template; and
(c) cleaving the product of the RCA reaction at the cleavage domains to release the ssDNA.

15. The method of claim 14, wherein all of the ssDNA molecules provided in (a) are produced in a single reaction mixture.

16. The method of claim 14 or 15, wherein the circular DNA molecule comprises a plurality of nucleotide sequences each encoding a ssDNA molecule provided in (a) bordered by cleavage domains.

17. The method of any one of claims 1 to 16, wherein the dsDNA molecule is linear.

18. The method of any one of claims 1 to 17 further comprising a step of amplifying the dsDNA molecule.

19. The method of claim 18, wherein the step of amplifying the dsDNA molecule comprises:
(i) inserting the dsDNA molecule into a nucleic acid vector (e.g. a DNA plasmid);
(ii) amplifying the vector (e.g. DNA plasmid);
(iii) excising the dsDNA molecule from the vector; and optionally
(iv) separating or purifying the dsDNA molecule, e.g. from the vector nucleic acid.

20. The method of claim 19, wherein step (ii) comprises transfecting the vector (e.g. DNA plasmid) into bacteria and growing the bacteria.

21. The method of any one of claims 1 to 20 further comprising a step of isolating the dsDNA molecule.

22. The method of any one of claims 1 to 21 further comprising a step of modifying the dsDNA molecule.

23. The method of any one of claims 1 to 22, wherein a plurality of versions of one or more of the plurality of ssDNA molecules is provided.

24. The method of claim 23, wherein the plurality of versions of one or more of the plurality of ssDNA molecules are sequence variants.

25. The method of claim 23 or 24, wherein the plurality of versions of one or more of the plurality of ssDNA molecules contain one or more modified (e.g. functionalised) nucleotides, wherein the versions differ with respect to: (i) the location and/or number of modified (e.g. functionalised) nucleotides; and/or (ii) the type of functional groups on the one or more modified nucleotides.

26. The method of any one of claims 1 to 22, wherein one or more of the plurality of ssDNA molecules contains one or more modified (e.g. functionalised) nucleotides.

27. The method of any one of claim 1 to 22 or 26, wherein each copy of a ssDNA molecule that provides a non-overlapping portion of a strand of the dsDNA molecule is identical.

28. A library of dsDNA molecules comprising a plurality of different dsDNA molecules obtained from the method of any one of claims 23 to 25.

29. Use of a plurality of single stranded DNA (ssDNA) molecules in a ligase-mediated ligation reaction to produce a double stranded DNA (dsDNA) molecule comprising at least 1500 bp, wherein:
(i) each of a first plurality of single stranded DNA (ssDNA) molecules comprises (e.g. consists of) a non-overlapping portion of a first strand of the dsDNA molecule, wherein a first ssDNA molecule comprises the 5′ end of the first strand of the dsDNA molecule and a second ssDNA: (a) comprises the 3′ end of the first strand of the dsDNA molecule or (b) is capable of providing (provides) the 3′ end of the first strand of the dsDNA molecule via a polymerase-mediated extension reaction using a ssDNA molecule comprising the 5′ end of the second strand as a template; and
(ii) each of a second plurality of ssDNA molecules comprises (e.g. consists of) a non-overlapping portion of a second strand of the dsDNA molecule, wherein a first ssDNA molecule comprises the 5′ end of the second strand of the dsDNA molecule and, optionally, a second ssDNA comprises the 3′ end of the second strand of the dsDNA molecule,
wherein:
(1) at least two of the plurality of ssDNA molecules from (ii) each comprise a first region that is fully complementary to an end region of a ssDNA molecule of (i) and a second region that is fully complementary to an end region of a different ssDNA molecule of (i);
(2) the ssDNA molecules of (i) to which the ssDNA molecules of (ii) are complementary form adjacent portions of the first strand of the dsDNA molecule;
(3) the regions that are complementary comprise at least 50 nucleotides;
(4) all of the ssDNA molecules comprise at least 150 nucleotides; and
(5) the ssDNA molecules are provided in equimolar amounts;
and wherein when the first and second pluralities of ssDNA molecules are contacted under conditions suitable for hybridisation of DNA molecules they form a dsDNA complex comprising all of the ssDNA molecules that can be ligated in a ligase-mediated reaction to produce the dsDNA molecule.

30. The use of claim 29, wherein:
(i) the ssDNA molecules are as defined in any one of claims 6 to 8, 12 to 16 or 23 to 27;
(ii) the regions that are complementary are as defined in claim 9; and/or
(iii) the dsDNA molecule is as defined in any one of claim 10, 11 or 17.
